(19) United States
(12) Patent Application Publication
(10) Pub. No.: US 2024/0073401 A1
(43) Pub. Date: Feb. 29, 2024

(54) EYEPIECE IMAGING ASSEMBLIES FOR A HEAD MOUNTED DISPLAY

(71) Applicant: Magic Leap, Inc., Plantation, FL (US)

(72) Inventors: Zhiheng JIA, Weston, FL (US); David COHEN, Nesher (IL); Lionel Ernest EDWIN, Hollywood, FL (US); Charles Robert SCHABACKER, Plantation, FL (US)

(21) Appl. No.: 18/259,044

(22) PCT Filed: Dec. 22, 2021
(86) PCT No.: PCT/US2021/064925

§ 371 Date: Jun. 22, 2023

Related U.S. Application Data
(60) Provisional application No. 63/130,274, filed on Dec. 23, 2020.

(51) Int. Cl. H04N 13/344; G02B 5/28; G02B 25/00; G02B 27/00; G02B 27/01

(52) U.S. Cl. CPC H04N 13/344; G02B 5/281; G02B 25/001; G02B 27/0093; G02B 27/0172; G02B 2027/0178

(57) ABSTRACT

A head mounted display can include a frame, an eyepiece, an image injection device, a sensor array, a reflector, and an off-axis optical element. The frame can be configured to be supported on the head of the user. The eyepiece can be coupled to the frame and configured to be disposed in front of an eye of the user. The eyepiece can include a plurality of layers. The image injection device can be configured to provide image content to the eyepiece for viewing by the user. The sensor array can be integrated in or one the eyepiece. The reflector can be disposed in or on the eyepiece and configured to reflect light received from an object for imaging by the sensor array. The off-axis optical element can be disposed in or one the eyepiece. The off-axis optical element can be configured to receive light reflected from the reflector and direct at least a portion of the light toward the sensor array.

### CROSS-REFERENCE TO RELATED APPLICATIONS

This application claims the benefit of priority to U.S. Provisional Application No. 63/130,274 (Attorney Docket No. MLEAP.317PR), filed Dec. 23, 2020, entitled “EYEPIECE IMAGING ASSEMBLIES FOR A HEAD MOUNTED DISPLAY.” This application is related to U.S. patent application Ser. No. 15/271,802 (Attorney Docket No. MLEAP.011A2), filed Sep. 21, 2016, entitled “EYE IMAGING WITH AN OFF-AXIS IMAGER,” U.S. patent application Ser. No. 15/925,505 (Attorney Docket No. MLEAP.099A3), filed Mar. 19, 2018, entitled “EYE-IMAGING APPARATUS USING DIFFRACTIVE OPTICAL ELEMENTS,” and International Application PCT/US2020/044107 (Attorney Docket No. MLEAP.247W0), filed Jul. 29, 2020, entitled “ANGULARLY SEGMENTED HOT MIRROR FOR EYE TRACKING.” The entirety of each application referenced in this paragraph is incorporated herein by reference.

### FIELD

The present disclosure relates to virtual reality and augmented reality imaging and visualization systems and in particular to imaging assemblies integrated in or on an eyepiece of a head mounted display.

### BACKGROUND

Modern computing and display technologies have facilitated the development of systems for so called “virtual reality” or “augmented reality” experiences, wherein digitally reproduced images or portions thereof are presented to a user in a manner wherein they seem to be, or may be perceived as, real. A virtual reality, or “VR”, scenario typically involves presentation of digital or virtual image information without transparency to other actual real-world visual input; an augmented reality, or “AR”, scenario typically involves presentation of digital or virtual image information as an augmentation to visualization of the actual world around the user. A mixed reality, or “MR”, scenario is a type of AR scenario and typically involves virtual objects that are integrated into, and responsive to, the natural world. For example, in an MR scenario, AR image content may be blocked by or otherwise be perceived as interacting with objects in the real world.

Referring to FIG. 1, an augmented reality scene 10 is depicted wherein a user of an AR technology sees a real-world park-like setting 20 featuring people, trees, buildings in the background, and a concrete platform 30. In addition to these items, the user of the AR technology also perceives that he “sees” “virtual content” such as a robot statue 40 standing upon the real-world platform 30, and a cartoon-like avatar character 50 flying by which seems to be a personification of a bumble bee, even though these elements 40, 50 do not exist in the real world. Because the human visual perception system is complex, it is challenging to produce an AR technology that facilitates a comfortable, natural-feeling, rich presentation of virtual image elements amongst other virtual or real-world imagery elements.

Systems and methods disclosed herein address various challenges related to AR and VR technology.

### SUMMARY

Various implementations of methods and apparatus within the scope of the appended claims each have several aspects, no single one of which is solely responsible for the desirable attributes described herein. Without limiting the scope of the appended claims, some prominent features are described herein.

One aspect of the present disclosure provides imaging an object such as an eye with an imaging assembly that does not directly view the object from a position straight in front of the object. Rather in various designs discussed herein, light from the object is reflected from an at least partially reflective optical element or reflector to a sensor. This reflector may be partially reflective and partially transmissive or transparent such that the user can see through the partially reflective/transmissive optical element. Accordingly, various optical devices according to various implementations described herein include such a reflector to direct light from an object to an imaging assembly so as to capture an image of the object as if the imaging assembly was in a position straight in front of the object. Various implementations described herein, for example, are configured to direct light from an eye to an imaging assembly so as to capture an image of the eye as if the imaging camera or sensor was in a position straight in front of the eye but instead with the sensor offset from the line-of-sight (e.g., straight forward line-of-sight), field-of-view of the eye (e.g., a central field-of-view) of the user's eye or any combination of these. Advantages of certain implementations include imaging objects as if viewing from a position straight in front of the object, but without interfering and/or obstructing the user's view of objects in directly front of the viewer (e.g., by placement of the imaging assembly temporally with respect to the eye). In various instances, the imaging assembly is integrated in or on the eyepiece of a head mounted display. Advantages of some such designs include a reduced camera form factor and increased alignment between optical components in or on the eyepiece. The imaging assemblies can be used to image the user's eye or an object in the environment.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 illustrates a user's view of augmented reality (AR) through an AR device.

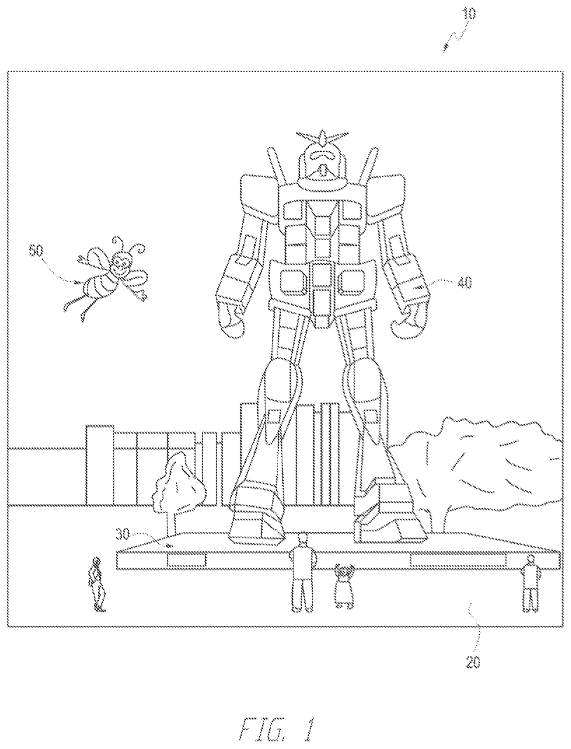

FIG. 2 illustrates an example of a wearable display system.

FIG. 3 illustrates a conventional display system for simulating three-dimensional imagery for a user.

FIG. 4 illustrates aspects of an approach to simulate three-dimensional imagery using multiple depth planes.

FIGS. 5A-5C illustrate relationships between radius of curvature and focal radius.

FIG. 6 illustrates an example of a waveguide stack to output image information to a user.

FIG. 7 illustrates an example of exit beams outputted by a waveguide.

FIG. 8 illustrates an example of a stacked waveguide assembly in which each depth plane includes images formed using multiple different component colors.

FIG. 9A illustrates a cross-sectional side view of an example of a set of stacked waveguides that each includes an in-coupling optical element.

FIG. 9B illustrates a perspective view of an example of the plurality of stacked waveguides of FIG. 9A.

FIG. 9C illustrates a top-down plan view of an example of the plurality of stacked waveguides of FIGS. 9A and 9B.

FIGS. 10A-10B schematically illustrate example imaging systems disposed so as not to obstruct a central field-of-view or straight forward line of sight of the user's eye comprising a partially transmissive, partially reflective optical element and a camera assembly to track an eye.

FIGS. 11A and 11B schematically illustrate example imaging systems disposed so as not to obstruct a central field-of-view or straight forward line of sight of the user's eye integrated in or on an eyepiece and configured to image the eye or an object in the environment, respectively.

FIGS. 12A and 12B schematically illustrate example sensor arrays and optical lenses integrated in or on an eyepiece with the lens on the same layer but opposite sides of the layer or with the lens and the sensor array on different layers than the sensor array.

FIG. 13 schematically illustrates another example imaging system with a sensor array having wafer scale optics integrated on a layer of an eyepiece.

FIG. 14 is a process flow diagram of an example of a method of imaging an object using a camera disposed so as not to obstruct a central field-of-view or straight forward line of sight of the user's eye.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

### Overview

A head mounted display (HMD) might use information about the state of the eyes of the wearer for a variety of purposes. For example, this information can be used for estimating the gaze direction of the wearer, for biometric identification, vision research, evaluate a physiological state of the wearer, etc. However, imaging the eyes can be challenging. The distance between the HMD and the wearer's eyes is short. Furthermore, gaze tracking requires a large field of view (FOV), while biometric identification requires a relatively high number of pixels on target on the iris. For imaging systems that seek to accomplish both of these objectives, these requirements are largely at odds. Furthermore, both problems may be further complicated by occlusion by the eyelids and eyelashes. Some current implementations for tracking eye movement use cameras mounted on the HMD and pointed directly toward the eye to capture direct images of the eye. However, in order to achieve the desired FOV and pixel number, the cameras are mounted within the wearer's FOV, thus tend to obstruct and interfere with the wearer's ability to see the surrounding world. Other implementations move the camera away from obstructing the wearer's view while directly imaging the eye, which results in imaging the eye from a high angle causing distortions of the image and reducing the field of view available for imaging the eye. Similarly, imaging objects in the environment, such as in front of the wearer's eyes, using cameras pointed directly toward the object may obstruct the wearer's view if aligned with the straight-forward line-of-sight or central field-of-view of the wearer. In addition, moving the camera away and/or orienting the camera at a large angle with respect to the object may cause distortions in the image of the object and/or provide a perspective different from that of the eye.

Implementations of the imaging systems described herein address some or all of these problems. Various implementations described herein, for instance, provide apparatus and systems capable of imaging an object (e.g., at least part of an eye, a portion of tissue surrounding an eye, or an object in the environment in front of a wearer's eye) while permitting the wearer to view the surrounding world. For example, an imaging system can comprise a sensor array integrated in or on the eyepiece disposed in front of an eye of the user. The eyepiece can include one or more optical elements (e.g., a reflector that is partially reflecting and partially transmitting and a transmissive diffractive optical element or refractive optical element that redirects the light toward the sensor array), configured to direct light from the object into the sensor array. The sensor array can receive at least a portion of the light, thus capture an image of the object as if in a direct view position from a distant position directly in front of the object such as the eye.

In some implementations, the imaging systems described herein may be a portion of display optics of an HMD (or a lens in a pair of eyeglasses or other eyewear). One or more reflective optical elements may be selected to reflect a first range of wavelengths while permitting unhindered propagation of a second range of wavelengths (for example, a range of wavelengths different from the first range) through the eyepiece. The first range of wavelengths can, for example, be in the infrared (IR), and the second range of wavelengths can be in the visible. For example, the eyepiece can comprise a reflective optical element (e.g., a reflector), which reflects IR light while transmitting visible light. In effect, the imaging system acts as if there were a virtual camera assembly directed back toward the object (e.g., wearer's eye). Thus, the virtual camera assembly can form an image using IR light reflected from the object (e.g., wearer's eye), while visible light from the surrounding world can be transmitted through the eyepiece and can be perceived by the wearer.

Without subscribing to any particular scientific theory, the embodiments described herein may include several non-limiting advantages. Several embodiments are capable of increasing the physical distance between the camera assembly and the eye, which may facilitate positioning the camera assembly out of the field of view or a central field-of-view of the wearer and therefore not obstructing the wearer's view such as the wearer's straightforward view while permitting capturing of a image of the eye equivalent to a direct view of the eye. Some of the embodiments described herein also may be configured to permit eye tracking using a larger field of view than conventional systems thus allowing eye tracking over a wide range of positions. Some implementations described herein also may be configured to image an object in the environment in front of the wearer's eye without obstructing the wearer's view. The use of IR imaging, for example, may facilitate imaging the eye without interfering with the wearer's ability to see through the eyepiece and view the environment.

Reference will now be made to the figures, in which like reference numerals refer to like parts throughout.

### Example HMD Device

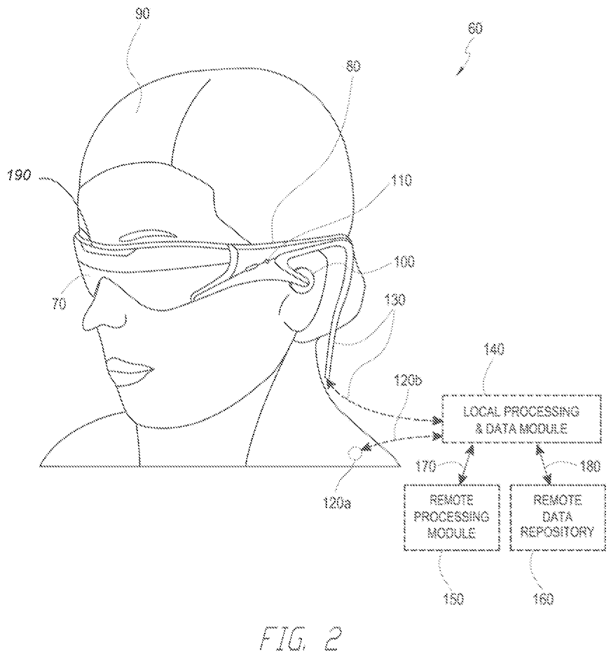

FIG. 2 illustrates an example of wearable display system 60. The display system 60 includes a display 70 comprising, for example, an eyepiece, and various mechanical and electronic modules and systems to support the functioning of that display 70. The display 70 may be coupled to a frame 80, which is wearable by a display system user or viewer 90 and which is configured to position the display 70 in front of the eyes of the user 90. The display 70 may be considered eyewear in some embodiments. In some embodiments, a speaker 100 is coupled to the frame 80 and configured to be positioned adjacent the ear canal of the user 90 (in some embodiments, another speaker, not shown, is positioned adjacent the other ear canal of the user to provide stereo/shapeable sound control). In some embodiments, the display system may also include one or more microphones 110 or other devices to detect sound. In some embodiments, the microphone is configured to allow the user to provide inputs or commands to the system 60 (e.g., the selection of voice menu commands, natural language questions, etc.), and/or may allow audio communication with other persons (e.g., with other users of similar display systems. The microphone may further be configured as a peripheral sensor to collect audio data (e.g., sounds from the user and/or environment). In some embodiments, the display system may also include a peripheral sensor 120a, which may be separate from the frame 80 and attached to the body of the user 90 (e.g., on the head, torso, an extremity, etc. of the user 90). The peripheral sensor 120a may be configured to acquire data characterizing the physiological state of the user 90 in some embodiments. For example, the sensor 120a may be an electrode.

With continued reference to FIG. 2, the display 70 is operatively coupled by communications link 130, such as by a wired lead or wireless connectivity, to a local data processing module 140 which may be mounted in a variety of configurations, such as fixedly attached to the frame 80, fixedly attached to a helmet or hat worn by the user, embedded in headphones, or otherwise removably attached to the user 90 (e.g., in a backpack-style configuration, in a belt-coupling style configuration). Similarly, the sensor 120a may be operatively coupled by communications link 120b, e.g., a wired lead or wireless connectivity, to the local processor and data module 140. The local processing and data module 140 may comprise a hardware processor, as well as digital memory, such as non-volatile memory (e.g., flash memory or hard disk drives), both of which may be utilized to assist in the processing, caching, and storage of data. The data may include data a) captured from sensors (which may be, e.g., operatively coupled to the frame 80 or otherwise attached to the user 90), such as image capture devices (such as, for example, cameras), microphones, inertial measurement units, accelerometers, compasses, GPS units, radio devices, gyros, and/or other sensors disclosed herein; and/or b) acquired and/or processed using remote processing module 150 and/or remote data repository 160 (including data relating to virtual content), possibly for passage to the display 70 after such processing or retrieval. The local processing and data module 140 may be operatively coupled by communication links 170, 180, such as via a wired or wireless communication links, to the remote processing module 150 and remote data repository 160 such that these remote modules 150, 160 are operatively coupled to each other and available as resources to the local processing and data module 140. In some embodiments, the local processing and data module 140 may include one or more of the image capture devices, microphones, inertial measurement units, accelerometers, compasses, GPS units, radio devices, and/or gyros. In some other embodiments, one or more of these sensors may be attached to the frame 80, or may be standalone structures that communicate with the local processing and data module 140 by wired or wireless communication pathways.

With continued reference to FIG. 2, in some embodiments, the remote processing module 150 may comprise one or more processors configured to analyze and process data and/or image information. In some embodiments, the remote data repository 160 may comprise a digital data storage facility, which may be available through the internet or other networking configuration in a “cloud” resource configuration. In some embodiments, the remote data repository 160 may include one or more remote servers, which provide information, e.g., information for generating augmented reality content, to the local processing and data module 140 and/or the remote processing module 150. In some embodiments, all data is stored and all computations are performed in the local processing and data module, allowing fully autonomous use from a remote module.

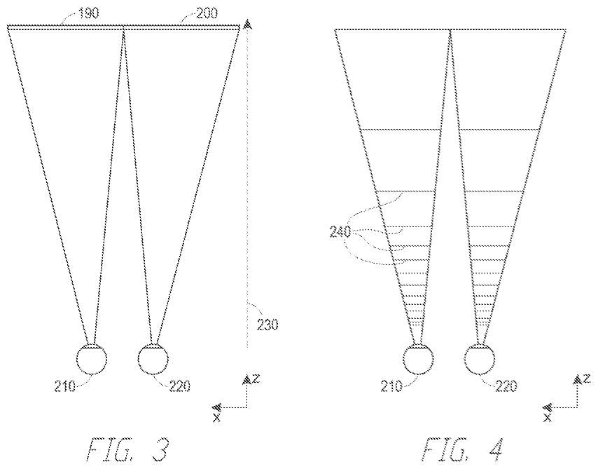

The perception of an image as being “three-dimensional” or “3-D” may be achieved by providing slightly different presentations of the image to each eye of the viewer. FIG. 3 illustrates a conventional display system for simulating three-dimensional imagery for a user. Two distinct images 190, 200—one for each eye 210, 220—are outputted to the user. The images 190, 200 are spaced from the eyes 210, 220 by a distance 230 along an optical or z-axis that is parallel to the line of sight of the viewer. The images 190, 200 are flat and the eyes 210, 220 may focus on the images by assuming a single accommodated state. Such 3-D display systems rely on the human visual system to combine the images 190, 200 to provide a perception of depth and/or scale for the combined image.

It will be appreciated, however, that the human visual system is more complicated and providing a realistic perception of depth is more challenging. For example, many viewers of conventional “3-D” display systems find such systems to be uncomfortable or may not perceive a sense of depth at all. Without being limited by theory, it is believed that viewers of an object may perceive the object as being “three-dimensional” due to a combination of vergence and accommodation. Vergence movements (e.g., rotation of the eyes so that the pupils move toward or away from each other to converge the lines of sight of the eyes to fixate upon an object) of the two eyes relative to each other are closely associated with focusing (or “accommodation”) of the lenses and pupils of the eyes. Under normal conditions, changing the focus of the lenses of the eyes, or accommodating the eyes, to change focus from one object to another object at a different distance will automatically cause a matching change in vergence to the same distance, under a relationship known as the “accommodation-vergence reflex,” as well as pupil dilation or constriction. Likewise, a change in vergence will trigger a matching change in accommodation of lens shape and pupil size, under normal conditions. As noted herein, many stereoscopic or “3-D” display systems display a scene using slightly different presentations (and, so, slightly different images) to each eye such that a three-dimensional perspective is perceived by the human visual system. Such systems are uncomfortable for many viewers, however, since they, among other things, simply provide a different presentation of a scene, but with the eyes viewing all the image information at a single accommodated state, and work against the “accommodation-vergence reflex.” Display systems that provide a better match between accommodation and vergence may form more realistic and comfortable simulations of three-dimensional imagery contributing to increased duration of wear and in turn compliance to diagnostic and therapy protocols.

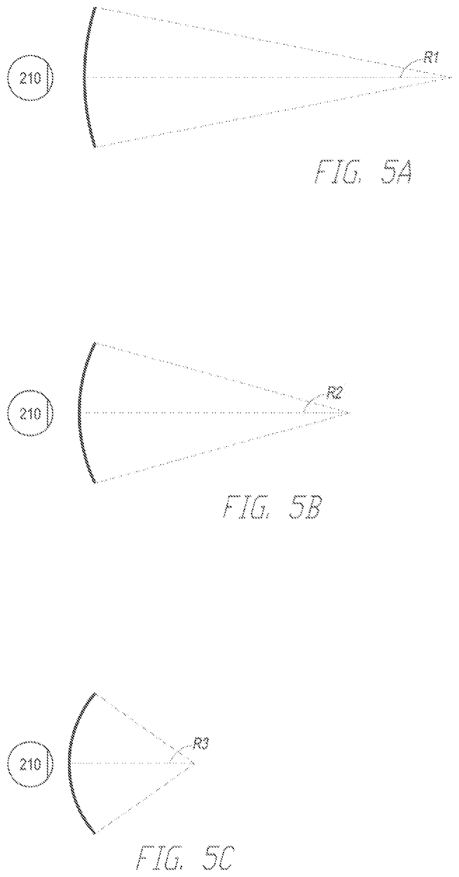

FIG. 4 illustrates aspects of an approach to simulate three-dimensional imagery using multiple depth planes. With reference to FIG. 4, objects at various distances from eyes 210, 220 on the z-axis are accommodated by the eyes 210, 220 so that those objects are in focus. The eyes 210, 220 assume particular accommodated states to bring into focus objects at different distances along the z-axis. Consequently, a particular accommodated state may be said to be associated with a particular one of depth planes 240, which has an associated focal distance, such that objects or parts of objects in a particular depth plane are in focus when the eye is in the accommodated state for that depth plane. In some embodiments, three-dimensional imagery may be simulated by providing different presentations of an image for each of the eyes 210, 220, and also by providing different presentations of the image corresponding to each of the depth planes. While shown as being separate for clarity of illustration, it will be appreciated that the fields of view of the eyes 210, 220 may overlap, for example, as distance along the z-axis increases. In addition, while shown as flat for ease of illustration, it will be appreciated that the contours of a depth plane may be curved in physical space, such that all features in a depth plane are in focus with the eye in a particular accommodated state.

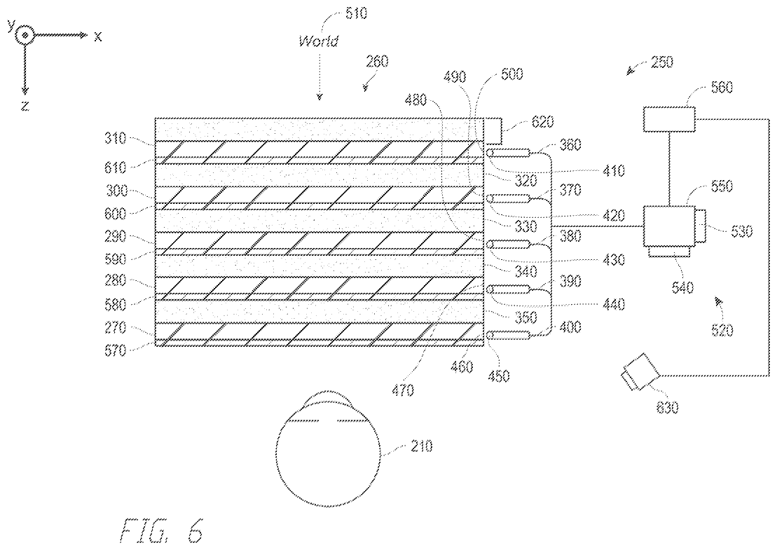

The distance between an object and the eye 210 or 220 may also change the amount of divergence of light from that object, as viewed by that eye. FIGS. 5A-5C illustrate relationships between distance and the divergence of light rays. The distance between the object and the eye 210 is represented by, in order of decreasing distance, R1, R2, and R3. As shown in FIGS. 5A-5C, the light rays become more divergent as distance to the object decreases. As distance increases, the light rays become more collimated. Stated another way, it may be said that the light field produced by a point (the object or a part of the object) has a spherical wavefront curvature, which is a function of how far away the point is from the eye of the user. The curvature increases with decreasing distance between the object and the eye 210. Consequently, at different depth planes, the degree of divergence of light rays is also different, with the degree of divergence increasing with decreasing distance between depth planes and the viewer's eye 210. While only a single eye 210 is illustrated for clarity of illustration in FIGS. 5A-5C and other figures herein, it will be appreciated that the discussions regarding eye 210 may be applied to both eyes 210 and 220 of a viewer.

Without being limited by theory, it is believed that the human eye typically can interpret a finite number of depth planes to provide depth perception. Consequently, a highly believable simulation of perceived depth may be achieved by providing, to the eye, different presentations of an image corresponding to each of these limited number of depth planes. The different presentations may be separately focused by the viewer's eyes, thereby helping to provide the user with depth cues based on the accommodation of the eye required to bring into focus different image features for the scene located on different depth plane and/or based on observing different image features on different depth planes being out of focus.

### Example of a Waveguide Stack Assembly

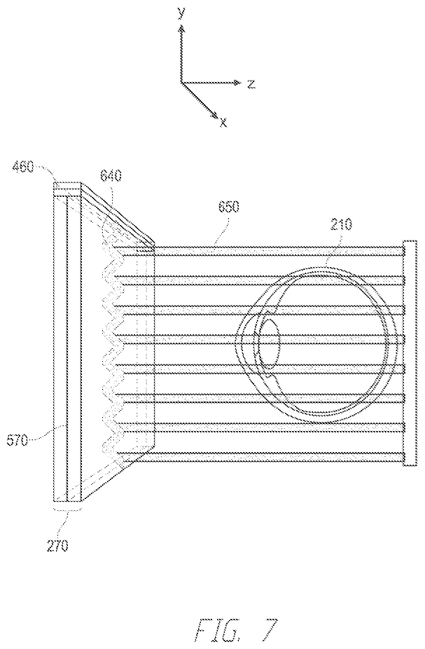

FIG. 6 illustrates an example of a waveguide stack to output image information to a user. A display system 250 includes a stack of waveguides, or stacked waveguide assembly, 260 that may be utilized to provide three-dimensional perception to the eye/brain using a plurality of waveguides 270, 280, 290, 300, 310. In some embodiments, the display system 250 is the system 60 of FIG. 2, with FIG. 6 schematically showing some parts of that system 60 in greater detail. For example, the waveguide assembly 260 may be part of the display 70 (e.g., form at least part of the eyepiece) of FIG. 2. It will be appreciated that the display system 250 may be considered a light field display in some embodiments.

With continued reference to FIG. 6, the waveguide assembly 260 may also include a plurality of features 320, 330, 340, 350 between the waveguides. In some embodiments, the features 320, 330, 340, 350 may be one or more lenses. The waveguides 270, 280, 290, 300, 310 and/or the plurality of lenses 320, 330, 340, 350 may be configured to send image information to the eye with various levels of wavefront curvature or light ray divergence. Each waveguide level may be associated with a particular depth plane and may be configured to output image information corresponding to that depth plane. Image injection devices 360, 370, 380, 390, 400 may function as a source of light for the waveguides and may be utilized to inject image information into the waveguides 270, 280, 290, 300, 310, each of which may be configured, as described herein, to distribute incoming light across each respective waveguide, for output toward the eye 210. Light exits an output surface 410, 420, 430, 440, 450 of the image injection devices 360, 370, 380, 390, 400 and is injected into a corresponding input surface 460, 470, 480, 490, 500 of the waveguides 270, 280, 290, 300, 310. In some embodiments, the each of the input surfaces 460, 470, 480, 490, 500 may be an edge of a corresponding waveguide, or may be part of a major surface of the corresponding waveguide (that is, one of the waveguide surfaces directly facing the world 510 or the viewer's eye 210). In some embodiments, a single beam of light (e.g. a collimated beam) may be injected into each waveguide to output an entire field of cloned collimated beams that are directed toward the eye 210 at particular angles (and amounts of divergence) corresponding to the depth plane associated with a particular waveguide. In some embodiments, a single one of the image injection devices 360, 370, 380, 390, 400 may be associated with and inject light into a plurality (e.g., three) of the waveguides 270, 280, 290, 300, 310.

In some embodiments, the image injection devices 360, 370, 380, 390, 400 are discrete displays that each produce image information for injection into a corresponding waveguide 270, 280, 290, 300, 310, respectively. In some other embodiments, the image injection devices 360, 370, 380, 390, 400 are the output ends of a single multiplexed display which may, e.g., pipe image information via one or more optical conduits (such as fiber optic cables) to each of the image injection devices 360, 370, 380, 390, 400. It will be appreciated that the image information provided by the image injection devices 360, 370, 380, 390, 400 may include light of different wavelengths, or colors (e.g., different component colors, as discussed herein).

In some embodiments, the light injected into the waveguides 270, 280, 290, 300, 310 is provided by a light projector system 520, which comprises a light module 530, which may include a light emitter, such as a light emitting diode (LED). The light from the light module 530 may be directed to and modified by a light modulator 540, e.g., a spatial light modulator, via a beam splitter 550. The light modulator 540 may be configured to change the perceived intensity of the light injected into the waveguides 270, 280, 290, 300, 310. Examples of spatial light modulators include liquid crystal displays (LCD) including a liquid crystal on silicon (LCOS) displays.

In some embodiments, the display system 250 may be a scanning fiber display comprising one or more scanning fibers configured to project light in various patterns (e.g., raster scan, spiral scan, Lissajous patterns, etc.) into one or more waveguides 270, 280, 290, 300, 310 and ultimately to the eye 210 of the viewer. In some embodiments, the illustrated image injection devices 360, 370, 380, 390, 400 may schematically represent a single scanning fiber or a bundle of scanning fibers configured to inject light into one or a plurality of the waveguides 270, 280, 290, 300, 310. In some other embodiments, the illustrated image injection devices 360, 370, 380, 390, 400 may schematically represent a plurality of scanning fibers or a plurality of bundles of scanning fibers, each of which are configured to inject light into an associated one of the waveguides 270, 280, 290, 300, 310. It will be appreciated that one or more optical fibers may be configured to transmit light from the light module 530 to the one or more waveguides 270, 280, 290, 300, and 310. It will be appreciated that one or more intervening optical structures may be provided between the scanning fiber, or fibers, and the one or more waveguides 270, 280, 290, 300, 310 to, e.g., redirect light exiting the scanning fiber into the one or more waveguides 270, 280, 290, 300, 310.

A controller 560 controls the operation of one or more of the stacked waveguide assembly 260, including operation of the image injection devices 360, 370, 380, 390, 400, the light source 530, and the light modulator 540. In some embodiments, the controller 560 is part of the local data processing module 140. The controller 560 includes programming (e.g., instructions in a non-transitory medium) that regulates the timing and provision of image information to the waveguides 270, 280, 290, 300, 310 according to, e.g., any of the various schemes disclosed herein. In some embodiments, the controller may be a single integral device, or a distributed system connected by wired or wireless communication channels. The controller 560 may be part of the processing modules 140 or 150 (FIG. 2) in some embodiments.

With continued reference to FIG. 6, the waveguides 270, 280, 290, 300, 310 may be configured to propagate light within each respective waveguide by TIR. The waveguides 270, 280, 290, 300, 310 may each be planar or have another shape (e.g., curved), with major top and bottom surfaces and edges extending between those major top and bottom surfaces. In the illustrated configuration, the waveguides 270, 280, 290, 300, 310 may each include out-coupling optical elements 570, 580, 590, 600, 610 that are configured to extract light out of a waveguide by redirecting the light, propagating within each respective waveguide, out of the waveguide to output image information to the eye 210. Extracted light may also be referred to as out-coupled light and the out-coupling optical elements light may also be referred to light extracting optical elements. An extracted beam of light may be outputted by the waveguide at locations at which the light propagating in the waveguide strikes a light extracting optical element. The out-coupling optical elements 570, 580, 590, 600, 610 may, for example, be gratings, including diffractive optical features, as discussed further herein. While illustrated disposed at the bottom major surfaces of the waveguides 270, 280, 290, 300, 310, for ease of description and drawing clarity, in some embodiments, the out-coupling optical elements 570, 580, 590, 600, 610 may be disposed at the top and/or bottom major surfaces, and/or may be disposed directly in the volume of the waveguides 270, 280, 290, 300, 310, as discussed further herein. In some embodiments, the out-coupling optical elements 570, 580, 590, 600, 610 may be formed in a layer of material that is attached to a transparent substrate to form the waveguides 270, 280, 290, 300, 310. In some other embodiments, the waveguides 270, 280, 290, 300, 310 may be a monolithic piece of material and the out-coupling optical elements 570, 580, 590, 600, 610 may be formed on a surface and/or in the interior of that piece of material.

With continued reference to FIG. 6, as discussed herein, each waveguide 270, 280, 290, 300, 310 is configured to output light to form an image corresponding to a particular depth plane. For example, the waveguide 270 nearest the eye may be configured to deliver collimated light (which was injected into such waveguide 270), to the eye 210. The collimated light may be representative of the optical infinity focal plane. The next waveguide up 280 may be configured to send out collimated light which passes through the first lens 350 (e.g., a negative lens) before it can reach the eye 210; such first lens 350 may be configured to create a slight convex wavefront curvature so that the eye/brain interprets light coming from that next waveguide up 280 as coming from a first focal plane closer inward toward the eye 210 from optical infinity. Similarly, the third up waveguide 290 passes its output light through both the first 350 and second 340 lenses before reaching the eye 210; the combined optical power of the first 350 and second 340 lenses may be configured to create another incremental amount of wavefront curvature so that the eye/brain interprets light coming from the third waveguide 290 as coming from a second focal plane that is even closer inward toward the person from optical infinity than was light from the next waveguide up 280.

The other waveguide layers 300, 310 and lenses 330, 320 are similarly configured, with the highest waveguide 310 in the stack sending its output through all of the lenses between it and the eye for an aggregate focal power representative of the closest focal plane to the person. To compensate for the stack of lenses 320, 330, 340, 350 when viewing/interpreting light coming from the world 510 on the other side of the stacked waveguide assembly 260, a compensating lens layer 620 may be disposed at the top of the stack to compensate for the aggregate power of the lens stack 320, 330, 340, 350 below. Such a configuration provides as many perceived focal planes as there are available waveguide/lens pairings. Both the out-coupling optical elements of the waveguides and the focusing aspects of the lenses may be static (i.e., not dynamic or electro-active). In some alternative embodiments, either or both may be dynamic using electro-active features.

In some embodiments, two or more of the waveguides 270, 280, 290, 300, 310 may have the same associated depth plane. For example, multiple waveguides 270, 280, 290, 300, 310 may be configured to output images set to the same depth plane, or multiple subsets of the waveguides 270, 280, 290, 300, 310 may be configured to output images set to the same plurality of depth planes, with one set for each depth plane. This can provide advantages for forming a tiled image to provide an expanded field of view at those depth planes.

With continued reference to FIG. 6, the out-coupling optical elements 570, 580, 590, 600, 610 may be configured to both redirect light out of their respective waveguides and to output this light with the appropriate amount of divergence or collimation for a particular depth plane associated with the waveguide. As a result, waveguides having different associated depth planes may have different configurations of out-coupling optical elements 570, 580, 590, 600, 610, which output light with a different amount of divergence depending on the associated depth plane. In some embodiments, the light extracting optical elements 570, 580, 590, 600, 610 may be volumetric or surface features, which may be configured to output light at specific angles. For example, the light extracting optical elements 570, 580, 590, 600, 610 may be volume holograms, surface holograms, and/or diffraction gratings. In some embodiments, the features 320, 330, 340, 350 may not be lenses; rather, they may simply be spacers (e.g., cladding layers and/or structures to form air gaps).

In some embodiments, the out-coupling optical elements 570, 580, 590, 600, 610 are diffractive features that form a diffraction pattern, or “diffractive optical element” (also referred to herein as a “DOE”). Preferably, the DOE's have a sufficiently low diffraction efficiency so that only a portion of the light of the beam is deflected away toward the eye 210 with each intersection of the DOE, while the rest continues to move through a waveguide via TIR. The light carrying the image information is thus divided into a number of related exit beams that exit the waveguide at a multiplicity of locations and the result is a fairly uniform pattern of exit emission toward the eye 210 for this particular collimated beam bouncing around within a waveguide.

In some embodiments, one or more DOEs may be switchable between “on” states in which they actively diffract, and “off” states in which they do not significantly diffract. For instance, a switchable DOE may comprise a layer of polymer dispersed liquid crystal, in which microdroplets comprise a diffraction pattern in a host medium, and the refractive index of the microdroplets may be switched to substantially match the refractive index of the host material (in which case the pattern does not appreciably diffract incident light) or the microdroplet may be switched to an index that does not match that of the host medium (in which case the pattern actively diffracts incident light).

In some embodiments, a camera assembly 630 (e.g., a digital camera, including visible light and IR light cameras) may be provided to capture images of the eye 210, parts of the eye 210, or at least a portion of the tissue surrounding the eye 210 to, e.g., detect user inputs, extract biometric information from the eye, estimate and track the gaze of the direction of the eye, to monitor the physiological state of the user, etc. As used herein, a camera may be any image capture device. In some embodiments, the camera assembly 630 may include an image capture device and a light source to project light (e.g., IR or near-IR light) to the eye, which may then be reflected by the eye and detected by the image capture device. In some embodiments, the light source includes light emitting diodes (“LEDs”), emitting in IR or near-IR. While the light source is illustrated as attached to the camera assembly 630, it will be appreciated that the light source may be disposed in other areas with respect to the camera assembly such that light emitted by the light source is directed to the eye of the wearer (e.g., light source 530 described herein). In some embodiments, the camera assembly 630 may be attached to the frame 80 (FIG. 2) and may be in electrical communication with the processing modules 140 or 150, which may process image information from the camera assembly 630 to make various determinations regarding, e.g., the physiological state of the user, the gaze direction of the wearer, iris identification, etc., as discussed herein. It will be appreciated that information regarding the physiological state of user may be used to determine the behavioral or emotional state of the user. Examples of such information include movements of the user or facial expressions of the user. The behavioral or emotional state of the user may then be triangulated with collected environmental or virtual content data so as to determine relationships between the behavioral or emotional state, physiological state, and environmental or virtual content data. In some embodiments, one camera assembly 630 may be utilized for each eye, to separately monitor each eye.

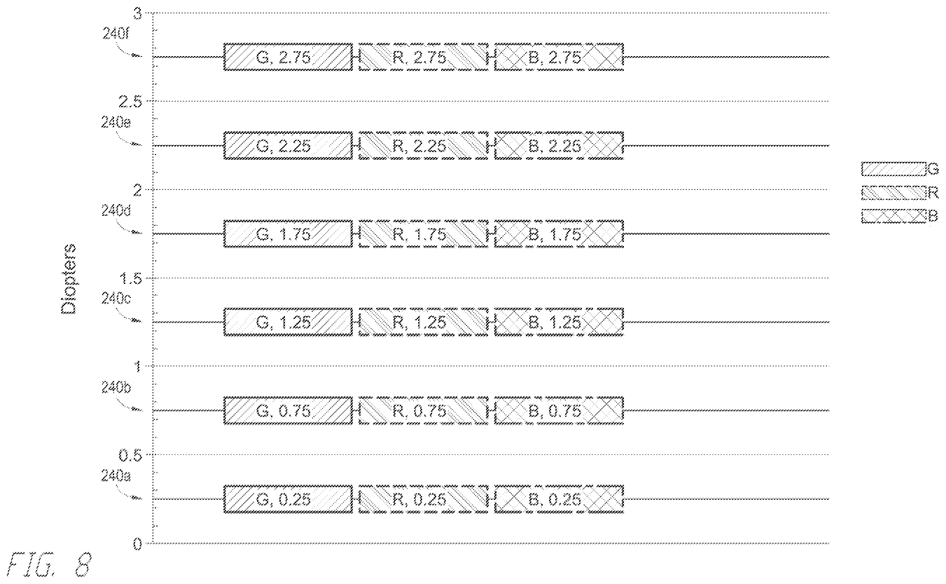

With reference now to FIG. 7, an example of exit beams outputted by a waveguide is shown. One waveguide is illustrated, but it will be appreciated that other waveguides in the waveguide assembly 260 (FIG. 6) may function similarly, where the waveguide assembly 260 includes multiple waveguides. Light 640 is injected into the waveguide 270 at the input surface 460 of the waveguide 270 and propagates within the waveguide 270 by TIR. At points where the light 640 impinges on the DOE 570, a portion of the light exits the waveguide as exit beams 650. The exit beams 650 are illustrated as substantially parallel but, as discussed herein, they may also be redirected to propagate to the eye 210 at an angle (e.g., forming divergent exit beams), depending on the depth plane associated with the waveguide 270. Substantially parallel exit beams may be indicative of a waveguide with out-coupling optical elements that out-couple light to form images that appear to be set on a depth plane at a large distance (e.g., optical infinity) from the eye 210. Other waveguides or other sets of out-coupling optical elements may output an exit beam pattern that is more divergent, which would require the eye 210 to accommodate to a closer distance to bring it into focus on the retina and would be interpreted by the brain as light from a distance closer to the eye 210 than optical infinity.

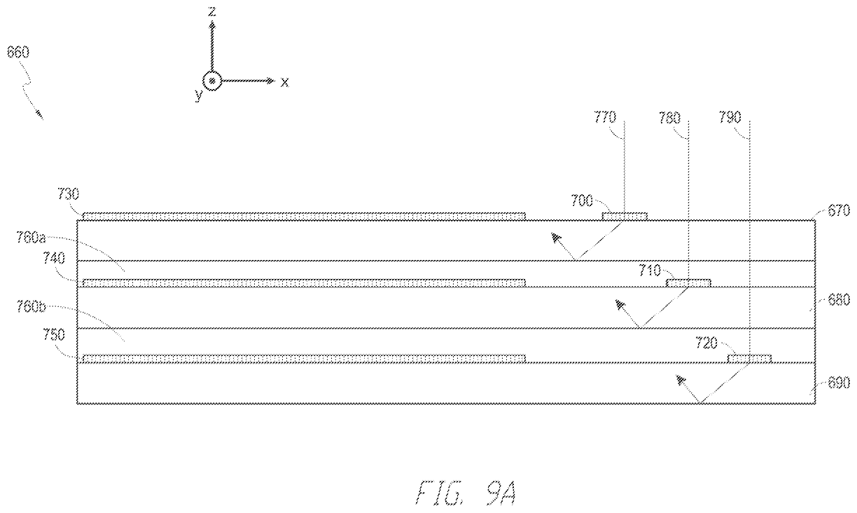

In some embodiments, a full color image may be formed at each depth plane by overlaying images in each of the component colors, e.g., three or more component colors. FIG. 8 illustrates an example of a stacked waveguide assembly in which each depth plane includes images formed using multiple different component colors. The illustrated embodiment shows depth planes 240a-240f, although more or fewer depths are also contemplated. Each depth plane may have three or more component color images associated with it, including: a first image of a first color, G; a second image of a second color, R; and a third image of a third color, B. Different depth planes are indicated in the figure by different numbers for diopters (dpt) following the letters G, R, and B. Just as examples, the numbers following each of these letters indicate diopters (1/m), or inverse distance of the depth plane from a viewer, and each box in the figures represents an individual component color image. In some embodiments, to account for differences in the eye's focusing of light of different wavelengths, the exact placement of the depth planes for different component colors may vary. For example, different component color images for a given depth plane may be placed on depth planes corresponding to different distances from the user. Such an arrangement may increase visual acuity and user comfort or may decrease chromatic aberrations.

In some embodiments, light of each component color may be outputted by a single dedicated waveguide and, consequently, each depth plane may have multiple waveguides associated with it. In such embodiments, each box in the figures including the letters G, R, or B may be understood to represent an individual waveguide, and three waveguides may be provided per depth plane where three component color images are provided per depth plane. While the waveguides associated with each depth plane are shown adjacent to one another in this drawing for ease of description, it will be appreciated that, in a physical device, the waveguides may all be arranged in a stack with one waveguide per level. In some other embodiments, multiple component colors may be outputted by the same waveguide, such that, e.g., only a single waveguide may be provided per depth plane.

With continued reference to FIG. 8, in some embodiments, G is the color green, R is the color red, and B is the color blue. In some other embodiments, other colors associated with other wavelengths of light, including magenta and cyan, may be used in addition to or may replace one or more of red, green, or blue. In some embodiments, features 320, 330, 340, and 350 may be active or passive optical filters configured to block or selectively pass light from the ambient environment to the viewer's eyes.

It will be appreciated that references to a given color of light throughout this disclosure will be understood to encompass light of one or more wavelengths within a range of wavelengths of light that are perceived by a viewer as being of that given color. For example, red light may include light of one or more wavelengths in the range of about 620-780 nm, green light may include light of one or more wavelengths in the range of about 492-577 nm, and blue light may include light of one or more wavelengths in the range of about 435-493 nm.

In some embodiments, the light source 530 (FIG. 6) may be configured to emit light of one or more wavelengths outside the visual perception range of the viewer, for example, IR or ultraviolet wavelengths. IR light can include light with wavelengths in a range from 700 nm to 10 μm. In some embodiments, IR light can include near-IR light with wavelengths in a range from 700 nm to 1.5 μm. In addition, the in-coupling, out-coupling, and other light redirecting structures of the waveguides of the display 250 may be configured to direct and emit this light out of the display towards the user's eye 210, e.g., for imaging or user stimulation applications.

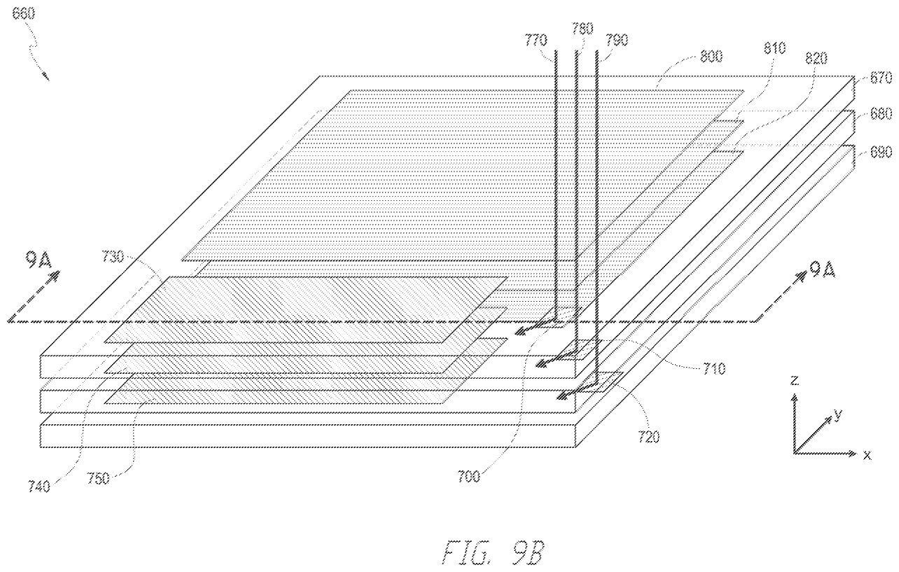

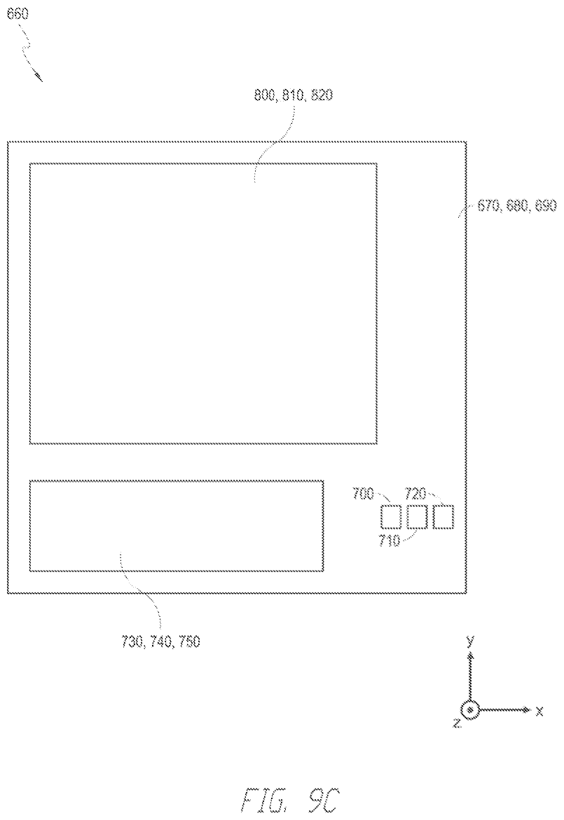

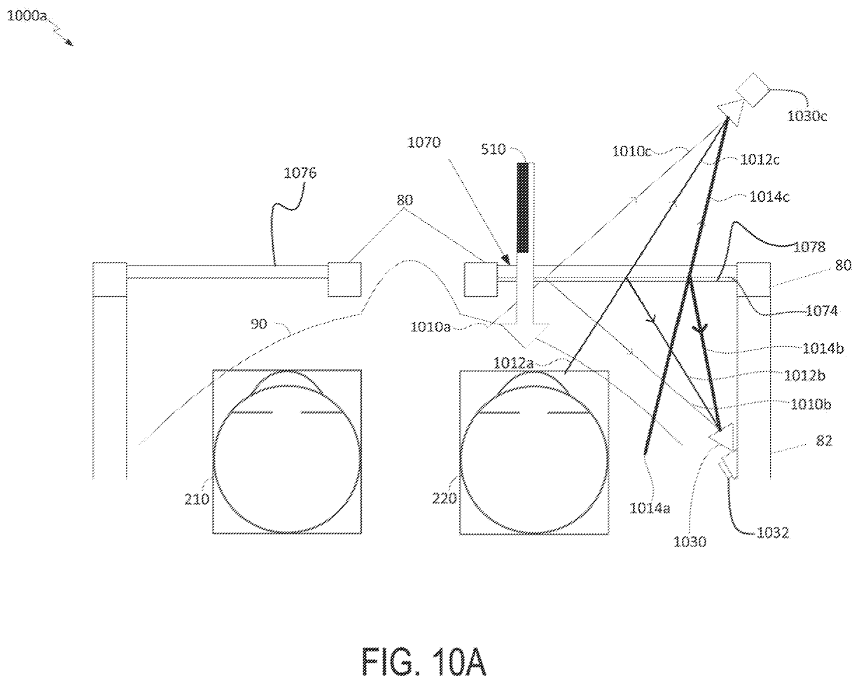

With reference now to FIG. 9A, in some embodiments, light impinging on a waveguide may need to be redirected to in-couple the light into the waveguide. An in-coupling optical element may be used to redirect and in-couple the light into its corresponding waveguide. FIG. 9A illustrates a cross-sectional side view of an example of a plurality or set 660 of stacked waveguides that each includes an in-coupling optical element. The waveguides may each be configured to output light of one or more different wavelengths, or one or more different ranges of wavelengths. It will be appreciated that the stack 660 may correspond to the stack 260 (FIG. 6) and the illustrated waveguides of the stack 660 may correspond to part of the plurality of waveguides 270, 280, 290, 300, 310, except that light from one or more of the image injection devices 360, 370, 380, 390, 400 is injected into the waveguides from a position that requires light to be redirected for in-coupling.

The illustrated set 660 of stacked waveguides includes waveguides 670, 680, and 690. Each waveguide includes an associated in-coupling optical element (which may also be referred to as a light input area on the waveguide), with, e.g., in-coupling optical element 700 disposed on a major surface (e.g., an upper major surface) of waveguide 670, in-coupling optical element 710 disposed on a major surface (e.g., an upper major surface) of waveguide 680, and in-coupling optical element 720 disposed on a major surface (e.g., an upper major surface) of waveguide 690. In some embodiments, one or more of the in-coupling optical elements 700, 710, 720 may be disposed on the bottom major surface of the respective waveguide 670, 680, 690 (particularly where the one or more in-coupling optical elements are reflective, deflecting optical elements). As illustrated, the in-coupling optical elements 700, 710, 720 may be disposed on the upper major surface of their respective waveguide 670, 680, 690 (or the top of the next lower waveguide), particularly where those in-coupling optical elements are transmissive, deflecting optical elements. In some embodiments, the in-coupling optical elements 700, 710, 720 may be disposed in the body of the respective waveguide 670, 680, 690. In some embodiments, as discussed herein, the in-coupling optical elements 700, 710, 720 are wavelength selective, such that they selectively redirect one or more wavelengths of light, while transmitting other wavelengths of light. While illustrated on one side or corner of their respective waveguide 670, 680, 690, it will be appreciated that the in-coupling optical elements 700, 710, 720 may be disposed in other areas of their respective waveguide 670, 680, 690 in some embodiments.

As illustrated, the in-coupling optical elements 700, 710, 720 may be laterally offset from one another. In some embodiments, each in-coupling optical element may be offset such that it receives light without that light passing through another in-coupling optical element. For example, each in-coupling optical element 700, 710, 720 may be configured to receive light from a different image injection device 360, 370, 380, 390, and 400 as shown in FIG. 6, and may be separated (e.g., laterally spaced apart) from other in-coupling optical elements 700, 710, 720 such that it substantially does not receive light from the other ones of the in-coupling optical elements 700, 710, 720.

Each waveguide also includes associated light distributing elements, with, e.g., light distributing elements 730 disposed on a major surface (e.g., a top major surface) of waveguide 670, light distributing elements 740 disposed on a major surface (e.g., a top major surface) of waveguide 680, and light distributing elements 750 disposed on a major surface (e.g., a top major surface) of waveguide 690. In some other embodiments, the light distributing elements 730, 740, 750 may be disposed on a bottom major surface of associated waveguides 670, 680, 690, respectively. In some other embodiments, the light distributing elements 730, 740, 750 may be disposed on both top and bottom major surface of associated waveguides 670, 680, 690 respectively; or the light distributing elements 730, 740, 750, may be disposed on different ones of the top and bottom major surfaces in different associated waveguides 670, 680, 690, respectively.

The waveguides 670, 680, 690 may be spaced apart and separated by, e.g., gas, liquid, or solid layers of material. For example, as illustrated, layer 760a may separate waveguides 670 and 680; and layer 760b may separate waveguides 680 and 690. In some embodiments, the layers 760a and 760b are formed of low refractive index materials (that is, materials having a lower refractive index than the material forming the immediately adjacent one of waveguides 670, 680, 690). Preferably, the refractive index of the material forming the layers 760a, 760b is 0.05 or more, or 0.10 or less than the refractive index of the material forming the waveguides 670, 680, 690. Advantageously, the lower refractive index layers 760a, 760b may function as cladding layers that facilitate TIR of light through the waveguides 670, 680, 690 (e.g., TIR between the top and bottom major surfaces of each waveguide). In some embodiments, the layers 760a, 760b are formed of air. While not illustrated, it will be appreciated that the top and bottom of the illustrated set 660 of waveguides may include immediately neighboring cladding layers.

Preferably, for ease of manufacturing and other considerations, the material forming the waveguides 670, 680, 690 are similar or the same, and the material forming the layers 760a, 760b are similar or the same. In some embodiments, the material forming the waveguides 670, 680, 690 may be different between one or more waveguides, or the material forming the layers 760a, 760b may be different, while still holding to the various refractive index relationships noted above.

With continued reference to FIG. 9A, light rays 770, 780, 790 are incident on the set 660 of waveguides. It will be appreciated that the light rays 770, 780, 790 may be injected into the waveguides 670, 680, 690 by one or more image injection devices 360, 370, 380, 390, 400 (FIG. 6).

In some embodiments, the light rays 770, 780, 790 have different properties, e.g., different wavelengths or different ranges of wavelengths, which may correspond to different colors. The in-coupling optical elements 700, 710, 720 each deflect the incident light such that the light propagates through a respective one of the waveguides 670, 680, 690 by TIR.

For example, in-coupling optical element 700 may be configured to deflect ray 770, which has a first wavelength or range of wavelengths. Similarly, the transmitted ray 780 impinges on and is deflected by the in-coupling optical element 710, which is configured to deflect light of a second wavelength or range of wavelengths. Likewise, the ray 790 is deflected by the in-coupling optical element 720, which is configured to selectively deflect light of third wavelength or range of wavelengths.

With continued reference to FIG. 9A, the deflected light rays 770, 780, 790 are deflected so that they propagate through a corresponding waveguide 670, 680, 690; that is, the in-coupling optical elements 700, 710, 720 of each waveguide deflects light into that corresponding waveguide 670, 680, 690 to in-couple light into that corresponding waveguide. The light rays 770, 780, 790 are deflected at angles that cause the light to propagate through the respective waveguide 670, 680, 690 by TIR. The light rays 770, 780, 790 propagate through the respective waveguide 670, 680, 690 by TIR until impinging on the waveguide's corresponding light distributing elements 730, 740, 750.

With reference now to FIG. 9B, a perspective view of an example of the plurality of stacked waveguides of FIG. 9A is illustrated. As noted above, the in-coupled light rays 770, 780, 790, are deflected by the in-coupling optical elements 700, 710, 720, respectively, and then propagate by TIR within the waveguides 670, 680, 690, respectively. The light rays 770, 780, 790 then impinge on the light distributing elements 730, 740, 750, respectively. The light distributing elements 730, 740, 750 deflect the light rays 770, 780, 790 so that they propagate towards the out-coupling optical elements 800, 810, and 820, respectively.

In some embodiments, the light distributing elements 730, 740, 750 are orthogonal pupil expanders (OPE's). In some embodiments, the OPE's both deflect or distribute light to the out-coupling optical elements 800, 810, 820 and also increase the beam or spot size of this light as it propagates to the out-coupling optical elements. In some embodiments, e.g., where the beam size is already of a desired size, the light distributing elements 730, 740, 750 may be omitted and the in-coupling optical elements 700, 710, 720 may be configured to deflect light directly to the out-coupling optical elements 800, 810, 820. For example, with reference to FIG. 9A, the light distributing elements 730, 740, 750 may be replaced with out-coupling optical elements 800, 810, 820, respectively. In some embodiments, the out-coupling optical elements 800, 810, 820 are exit pupils (EP's) or exit pupil expanders (EPE's) that direct light in a viewer's eye 210 (FIG. 7). It will be appreciated that the OPE's may be configured to increase the dimensions of the eye box in at least one axis and the EPE's may be to increase the eye box in an axis crossing, e.g., orthogonal to, the axis of the OPEs.

Accordingly, with reference to FIGS. 9A and 9B, in some embodiments, the set 660 of waveguides includes waveguides 670, 680, 690; in-coupling optical elements 700, 710, 720; light distributing elements (e.g., OPE's) 730, 740, 750; and out-coupling optical elements (e.g., EP's) 800, 810, 820 for each component color. The waveguides 670, 680, 690 may be stacked with an air gap/cladding layer between each one. The in-coupling optical elements 700, 710, 720 redirect or deflect incident light (with different in-coupling optical elements receiving light of different wavelengths) into its waveguide. The light then propagates at an angle that will result in TIR within the respective waveguide 670, 680, 690. In the example shown, light ray 770 (e.g., blue light) is deflected by the first in-coupling optical element 700, and then continues to bounce down the waveguide, interacting with the light distributing element (e.g., OPE's) 730 and then the out-coupling optical element (e.g., EPs) 800, in a manner described earlier. The light rays 780 and 790 (e.g., green and red light, respectively) will pass through the waveguide 670, with light ray 780 impinging on and being deflected by in-coupling optical element 710. The light ray 780 then bounces down the waveguide 680 via TIR, proceeding on to its light distributing element (e.g., OPEs) 740 and then the out-coupling optical element (e.g., EP's) 810. Finally, light ray 790 (e.g., red light) passes through the waveguide 690 to impinge on the light in-coupling optical elements 720 of the waveguide 690. The light in-coupling optical elements 720 deflect the light ray 790 such that the light ray propagates to light distributing element (e.g., OPEs) 750 by TIR, and then to the out-coupling optical element (e.g., EPs) 820 by TIR. The out-coupling optical element 820 then finally out-couples the light ray 790 to the viewer, who also receives the out-coupled light from the other waveguides 670, 680.

FIG. 9C illustrates a top-down plan view of an example of the plurality of stacked waveguides of FIGS. 9A and 9B. As illustrated, the waveguides 670, 680, 690, along with each waveguide's associated light distributing element 730, 740, 750 and associated out-coupling optical element 800, 810, 820, may be vertically aligned. However, as discussed herein, the in-coupling optical elements 700, 710, 720 are not vertically aligned; rather, the in-coupling optical elements are preferably non-overlapping (e.g., laterally spaced apart as seen in the top-down view). As discussed further herein, this non-overlapping spatial arrangement facilitates the injection of light from different resources into different waveguides on a one-to-one basis, thereby allowing a specific light source to be uniquely coupled to a specific waveguide. In some embodiments, arrangements including non-overlapping spatially separated in-coupling optical elements may be referred to as a shifted pupil system, and the in-coupling optical elements within these arrangements may correspond to sub pupils.

### Example Imaging Systems

The eyes or tissue around the eyes of the wearer of a HMD (e.g., the wearable display system 200 shown in FIG. 2) can be imaged using one or more optical elements to direct light from the eye into a camera assembly. The resulting images can be used to track an eye or eyes, image the retina, reconstruct the eye shape in three dimensions, extract biometric information from the eye (e.g., iris identification), etc.

As outlined above, there are a variety of reasons why a HMD might use information about the state of the eyes of the wearer. For example, this information can be used for estimating the gaze direction of the wearer or for biometric identification. This problem is challenging, however, because of the short distance between the HMD and the wearer's eyes. It is further complicated by the fact that gaze tracking requires a larger field of view, while biometric identification requires a relatively high number of pixels on target on the iris. For an imaging system that will attempt to accomplish both of these objectives, the requirements of the two tasks are largely at odds. Finally, both problems are further complicated by occlusion by the eyelids and eyelashes. Embodiments of the imaging systems described herein may address at least some or all of these problems.

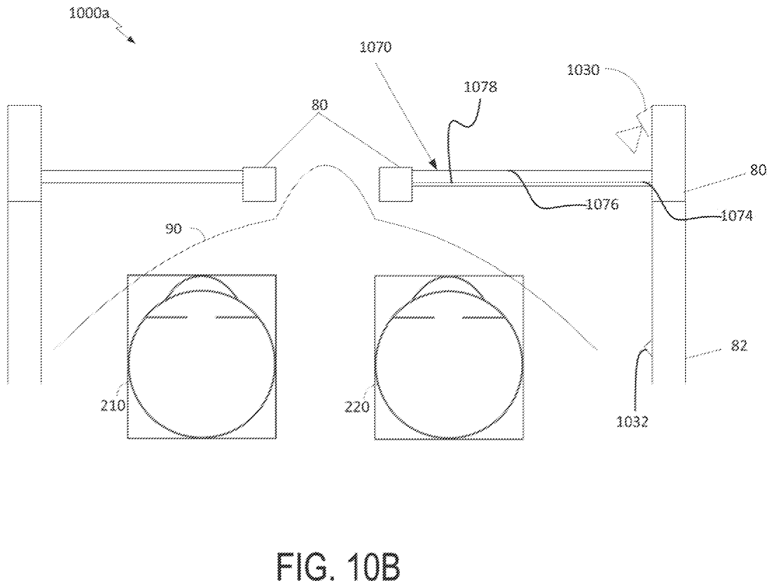

FIGS. 10A and 10B schematically illustrate examples of an imaging system 1000a configured to image one or both eyes 210, 220 of a wearer 90. The imaging system 1000a comprises a substrate 1070 and a camera assembly 1030 arranged to view the eye 220. Embodiments of the imaging system 1000a described herein with reference to FIGS. 10A and 10B can be used with HMDs including the display devices described herein (e.g., the wearable display system 60 shown in FIG. 2, the display system 250 shown in FIGS. 6 and 7, and the stack 660 of FIGS. 9A-9C). For example, in some implementations where the imaging system 1000a is part of the display system 250 of FIG. 6, the substrate 1070 may comprise or replace one of the waveguides 270, 280, 290, 300, or 310, be disposed between two of the waveguides 270, 280, 290, 300, or 310, may be disposed between the waveguide stack 260 and eye 210, or may be disposed between the waveguide stack 260 and the world 510.

In some embodiments, the camera assembly 1030 may be mounted in proximity to the wearer's eye, for example, on a frame 80 of the wearable display system 60 of FIG. 2 (e.g., on an ear stem 82 near the wearer's temple); around the edges of the display (e.g., eyepiece) 70 of FIG. 2 (as shown in FIG. 10B); and/or embedded in the display (e.g. eyepiece) 70 of FIG. 2. The camera assembly 1030 may be substantially similar to camera assembly 630 of FIG. 6. In other embodiments, a second camera assembly can be used to separately image the wearer's other eye 210. The camera assembly 1030 can include an IR digital camera that is sensitive to IR radiation. The camera assembly 1030 can be mounted so that it is forward facing (e.g., in the direction of the wearer's vision toward), as illustrated in FIG. 10A, or the camera assembly 1030 can be mounted to be facing backward and directed backward, for example. the eye 220 or eye side (e.g., FIG. 10B).

In some embodiments, the camera assembly 1030 may include an image capture device and a light source 1032 to project light to the eye 220, which may then be reflected by the eye 220 and detected by the camera assembly 1030. While the light source 1032 is illustrated as attached to the camera assembly 1030, the light source 1032 may be disposed in other areas with respect to the camera assembly such that light emitted by the light source is directed to the eye of the wearer and reflected to the camera assembly 1030. For example, where the imaging system 1000a is part of the display system 250 (FIG. 6) and the substrate 1070 comprises or replaces one of waveguides 270, 280, 290, 300, or 310, the light source 1032 may be one of light emitters 360, 370, 380, 390, or light source 530.

In the embodiment illustrated in FIG. 10A, the camera assembly 1030 is positioned to be directed toward the substrate such as a proximal surface 1074 of the substrate 1070. The substrate 1070 can be, for example, a portion of the display (e.g., eyepiece) 70 of FIG. 2 or a lens in a pair of eyeglasses. The substrate 1070 can be transmissive to at least 10%, 20%, 30%, 40%, 50%, or more of visible light incident on the substrate 1070 possible up to 80%, 90%, 95%, 98%, 99%, 99.9%, 99.99% or more or in any range formed by any of these values. In other embodiments, the substrate 1070 need not be transparent (e.g., in a virtual reality display). The substrate 1070 can comprise one or more reflective optical elements 1078. In some embodiments, one or more reflective optical elements 1078 may be selected to reflect a first range of wavelengths while being substantially transmissive to a second range of wavelengths different from the first range of wavelengths. In some embodiments, the first range of wavelengths can be IR wavelengths, and the second range of wavelengths can be visible wavelengths. Accordingly, in certain implementations, the one or more reflective optical elements may comprise a hot mirror or reflector 1078. The substrate 1070 may comprise a polymer or plastic material such as polycarbonate or other lightweight materials having the desired optical properties. Without subscribing to a particular scientific theory, plastic materials may be less rigid and thus less susceptible to breakage or defects during use. Plastic materials may also be lightweight, thus, when combined with the rigidity of the plastic materials allowing thinner substrates, may facilitate manufacturing of compact and light weight imaging systems. While the substrate 1070 is described as comprising a polymer such as polycarbonate or other plastic having the desired optical properties, other materials are possible, such as having the desired optical properties, for example, fused silica.

The one or more reflective optical elements 1078 can comprise a reflective optical element configured to reflect or redirect light of a first range of wavelengths (e.g., IR light) while transmitting light of a second range of wavelengths (e.g., visible light). In such embodiments, IR light 1010a, 1012a, and 1014a from the eye 220 reflects from one or more optical elements 1078, resulting in reflected IR light 1010b, 1012b, 1014b which can be used to form an image by the camera assembly 1030. In some embodiments, the camera assembly 1030 can be sensitive to or able to capture at least a subset (such as a non-empty subset or a subset of less than all) of the first range of wavelengths reflected by the one or more reflective optical elements 1078. For example, where the one or more reflective optical elements 1078 may be a partially transmissive reflective element, the one or more optical elements 1078 may reflect IR light in the a range of 700 nm to 1.5 μm, and the camera assembly 1030 may be sensitive to or able to capture near IR light at wavelengths in the range from 700 nm to 900 nm. As another example, the one or more reflective optical elements 1078 may reflect IR light in a range from 700 nm to 1.5 μm. In some implementations, the camera assembly 1030 may include a filter that filters out IR light in the range of 900 nm to 1.5 μm such that the camera assembly 1030 can capture near IR light at wavelengths from 700 nm to 900 nm.

Visible light from the outside world (e.g., world 510 of FIG. 6) can be transmitted through the substrate 1070 and perceived by the wearer. Nevertheless, light can be reflected from objects on the opposite side of the substrate (e.g., the eye side) and be incident on the camera assembly 1030. In effect, as a result of this reflection produced by the reflective optical element one or more optical elements 1078, the imaging system 1000a can act as if there were a virtual camera assembly 1030c directed back toward the wearer's eye 220 capturing a direct view image of the eye 220. Virtual camera assembly 1030c is labeled with reference to “c” because it may comprise a virtual image formed by virtual IR light rays 1010c, 1012c, and 1014c propagated from the wearer's eye 220 to the substrate 1070. Although the one or more reflective optical elements 1078 is illustrated as disposed on the proximal surface 1074 of the substrate 1070, other configurations are possible. For example, the one or more reflective optical elements 1078 can be disposed on a distal surface 1076 of the substrate 1060 or within the substrate 1070.

Accordingly, while example arrangements of imaging systems 1000a are shown in FIGS. 10A and 10B, other arrangements are possible.

### Example Imaging Systems Integrated in or on an Eyepiece

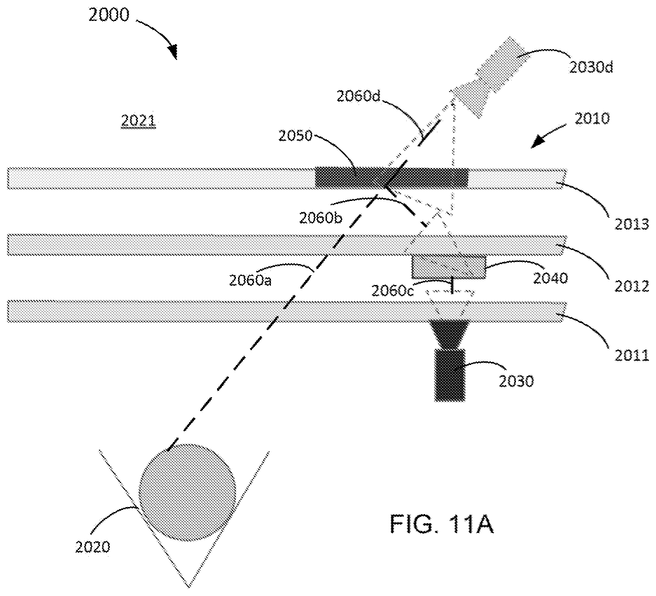

FIG. 11A schematically illustrates another example imaging system 2000 that can be used with HMDs (e.g., eyewear) including the display devices described herein (e.g., the wearable display system 60 shown in FIG. 2, the display system 250 shown in FIGS. 6 and 7, and the stack 660 of FIGS. 9A-9C). For example, in some implementations, the eyepiece 2010 shown in FIG. 11A can be coupled to a frame (e.g., frame 80 shown in FIG. 2), which can be configured to be supported on the head of the user. Additionally, the eyepiece 2010 can be configured to be disposed in front of an eye 2020 of the user. The eyepiece 2010 can comprise at least a portion that is transparent to transmit light from a portion of the environment 2021 in front of the eyepiece 2010 to the user's eye 2020 for viewing therethrough to see said environment in front of the eyepiece and the user's eye. The eyepiece 2010 can also provide virtual content to the user's eye 2020 for viewing. In various implementations, the eyepiece 2010 can include one or more layers (e.g., one or more of the waveguides 270, 280, 290, 300, 310 shown in FIG. 6). Further, one or more of the image injection devices (e.g., image injection devices 360, 370, 380, 390, 400 shown in FIG. 6) can be configured to provide image content to the eyepiece 2010 for viewing by the user. For example, the eyepiece 2010 can be configured to direct light from one or more image injection devices to the user's eye 2020 to present image content to the user.

The imaging system 2000 can also include a sensor array 2030, a refractive or transmissive diffractive optical element 2040, and a reflector 2050. In various implementations, the imaging system 2000 and/or various portions thereof (e.g., the sensor array 2030, refractive or transmissive diffractive optical element 2040, and reflector 2050) may be disposed or integrated in or on the eyepiece 2010. The reflector 2050 can be configured to reflect light 2060a received from an object (e.g., an eye 2020 of the user, a part of the eye 2020, or a portion of tissue surrounding the eye 2020) for imaging by the sensor array 2030. The reflector or reflective optical element 2050 may be partially reflective and partially transmissive such that the user can see therethrough to the environment in front of the user. The refractive or transmissive diffractive optical element 2040 can be configured to receive light 2060b reflected from the reflector 2050 and re-direct at least a portion of the light 2060c toward the sensor array 2030. The refractive or transmissive diffractive optical element 2040 may, for example, comprise a transmissive element such as a transmissive diffractive optical element or diffraction grating configured to transmit and diffract light from the reflector 2050 incident thereon so as to deflect and redirect at least a portion of said light to said the sensor array 2030. The refractive or transmissive diffractive optical element 2040 may also comprise a transmissive element such as a refractive optical element configured to refract light from the reflector 2050 incident thereon so as to deflect and redirect at least a portion of said light to said the sensor array 2030.

In some implementations, the refractive or transmissive diffractive optical element 2040 may comprise an off-axis optical element. Such an off-axis optical element may deflects a beam so as to be propagating parallel to or along an axis such as an optical axis of an optical element or optical system when the incident beam is directed in a different “askew” direction that does not propagate along that axis, or vice versa. In this instance, the optical axis may be the optical axis of the camera comprising the sensor array and may include the optical axis of the off-axis optical element. An off-axis optical element may have a mechanical center that is offset from the optical axis or center of rotational symmetry. In various implementations, the off-axis optical element is configured to have an axis, e.g., optical axis, that is normal to the off-axis optical element and/or the eyepiece 2010 or layer thereof on which said off-axis optical element is disposed in or on. This optical axis may correspond in some cases to an axis of symmetry for an optical surface or optical features included in or on the off-axis optical element. Light from the reflector 2050 can be deflected, diffracted, refracted, redirected, or any combination of these, so as to propagate more along said axis or optical axis toward said sensor array 2030.

As a result of the reflector, in effect, the imaging system 2000 can act as if there were a virtual camera assembly 2030d directed back toward the object 2020 capturing a direct view of the object 2020. Virtual camera assembly 2030d may be considered, for example, to image the object 2020 via virtual IR light rays 2060d propagated from the object through the eyepiece 2010.

As described herein, the eyepiece 2010 may comprise one or more layers. In FIG. 11A, three layers 2011, 2012, 2013 are shown. However, some implementations may include only one or two layers. Other implementations may include three or more layers (e.g., three, four, five, six, seven, eight, nine, ten, etc., or any ranges formed by such values). The layers 2011, 2012, 2013 may comprise one or more waveguides configured to guide light therein by total internal reflection. For example, the layers 2011, 2012, 2013 may include one or more of the waveguides (e.g., one or more of the waveguides 270, 280, 290, 310 shown in FIG. 6) configured to receive light from one or more of the image injection devices (e.g., one or more of the image injection devices 360, 370, 380, 390, 400 shown in FIG. 6). One or more waveguides may be configured to propagate light within by total internal reflection. One or more waveguides may include one or more in-coupling optical elements (e.g., 700, 710, 720 shown in FIG. 9A), out-coupling optical elements (e.g., 570, 580, 590, 600, 610 shown in FIG. 6), or other light redirecting optical elements (e.g., 730, 740, 750 shown in FIG. 9A) configured to distribute and/or extract light out of one or more waveguides to output image information to the user's eye 2020. In some implementations, the waveguides may comprise one or more compound pupil expanders (CPEs) configured to expand the pupil as well as output light guided within the waveguide. Any one or more of these in-coupling optical elements, out-coupling optical elements, redirect optical elements, and compound pupil expanders may comprise diffractive optical elements such as diffraction gratings or holographic optical elements. In some instances, different waveguides can be arranged to provide different color image content.

Other optical elements (for example, lenses, polarizers, prisms, etc.) may be used to manipulate, e.g., to focus, correct aberrations, direct, etc., light as desired for the specific application. For example, the eyepiece 2010 (e.g., one or more layers 2011, 2012, 2013) may include one or more lenses (e.g., lenses 320, 330, 340, 350 shown in FIG. 6) configured to direct image information to the eye 2020 with various levels of wavefront curvature or light ray divergence or lack thereof (e.g., as in a collimated beam). As described herein, the eyepiece 2010 may include different waveguides or groups of waveguides configured to project light into the user's eye 2020 to display image content with different amounts of divergences and/or collimation possibly so as to be diverging or collimated thereby providing light as if projected from different distances from the user's eye 2020. In some instances, the eyepiece 2010 may include a front or rear lens. For example, a compensating lens layer 620 as shown in FIG. 6 can be used to compensate for the aggregate power of lenses in the layers 2011, 2012, 2013. As another example, a lens can be used to extend the depth of focus. In some instances, a prescription lens can be used to provide for refractive correction for a user having a refractive deficiency. For example, a lens can be used to correct for myopia, hyperopia, presbyopia, astigmatism, etc. or any combination thereof.

In some implementations, the eyepiece 2010 (e.g., one or more layers 2011, 2012, 2013) may comprise an outer lens or window such as a decorative (or cosmetic) lens. For example, the outer or decorative lens may have a shape such as a curved shape that affects the appearance of the eyewear. In some implementations, the outer or decorative lens may be a lens for use as sunglasses to filter out sunlight. In another example, the outer or decorative lens may be a color filtering lens for use in goggles. In yet other examples, the outer or decorative lens may have a colored visual appearance that is viewable by other people who are not wearing the lens (e.g., a lens that appears blue, red, etc. to other people). The outer or decorative lens may also include a color layer that is viewed by people other than the user. In some instances, one or more lenses or surfaces can be provided with an anti-reflective and/or a scratch resistant coating.

In some implementations, the eyepiece 2010 (e.g., one or more layers 2011, 2012, 2013) can include an illumination layer and/or a dimmer. The illumination layer and/or dimmer may be used to adjust the brightness and/or contrast of the eyepiece 2010 and thus, of the projected images for viewing. As an example, the illumination layer can provide light (e.g., from one or more light sources) to illuminate the eye for example with infrared light to be imaged and/or to provide glints on the eye possibly for eye tracking. As another example, the dimmer can reduce the amount of light for the images to be viewed. In some instances, the dimmer may include one or more films (e.g., one or more rainbow films or filters to block certain wavelengths or amounts of light). In some instances, the dimmer may be a curved layer (e.g., a curved lens).

In some implementations, the eyepiece 2010 (e.g., one or more layers 2011, 2012, 2013) can comprise a material having the desired optical properties. As described herein, when disposed at a location in front of the user's eye 2020 when the user wears the HMD, the eyepiece 2010 can comprise a portion that transmits light from a portion of the environment 2021 in front of the user and the eyepiece 2010 to the user's eye 2020 to provide a view of the portion of the environment 2021. In various implementations, one or more of the layers 2011, 2012, 2013 of the eyepiece 2010 can include at least a portion thereof which is transparent. In some examples, the eyepiece 2010 can comprise at least one glass or plastic/polymer layer that is transparent to visible light. In some instances, the eyepiece 2010 can be transmissive and/or transparent to at least 10%, 20%, 30%, 40%, 50%, or more of visible light possible up to 80%, 90%, 95%, 98%, 99%, 99.9%, 99.99% or more or in any range formed by any of these values.

With continued reference to FIG. 11A, the imaging system 2000 can comprise a sensor array 2030 integrated in or on the eyepiece. The sensor array 2030 is shown on layer 2011, however, the sensor array 2030 can be integrated in or on any one or more of the layers 2011, 2012 2013 of the eyepiece 2010, e.g., in or on one or more of a waveguide, an illumination layer, a dimmer layer, a front lens, a rear lens, a cosmetic or decorative lens, etc. The sensor array 2030 can be configured to image an object. For example, the sensor array 2030 can be provided so that it is forward facing (e.g., in the direction of the wearer's vision field), as illustrated in FIG. 11A, and can be configured to view the wearer's eye 2020 (or a part of the eye 2020 or a portion of tissue surrounding the eye 2020) to capture images of the eye 2020 by receiving light from the eye reflected to the sensor array by the reflector 2050. As another example, as illustrated in FIG. 11B, the sensor array 2030 can be provided so that it is facing backward (e.g., facing a direction opposite that illustrated in FIG. 11A), and directed at the eye 2020 and can image an object 2022 in the environment 2021 in which the wearer is viewing by receiving light from the object reflected to the sensor array by the reflector 2050.

The sensor array 2030 can be integrated in or on the eyepiece 2010 at any position around the wearer's eye 2020 such that the sensor array 2030 does not obstruct the wearer's view of the surrounding world or central field-of-view or straight forward line of sight or impede the operation of the HMD. In some implementations, the sensor array 2030 can be integrated in or on the eyepiece 2010, e.g., adjacent an ear stem or temple, adjacent a nose bridge, above the eye, or below the eye and over the cheek. In some implementations, the sensor array 2030 can be integrated in or on the eyepiece 2010 at a location closer to an edge of the eyepiece or the temple or ear stem or the nose piece or any combination of these than the center of the eyepiece or the location on the eyepiece through which the straight forward line-of-sight is directed through. In some implementations, additional sensor arrays can be used for additional objects, such as the wearer's other eye so that each eye can be separately imaged, or other objects in the environment 2021 so that multiple objects can be separately imaged.

In some instances, the sensor array 2030 can include a CMOS detector array. In some instances, the sensor array 2030 can include a CCD detector array. In some examples, the sensor array 2030 can comprise detector pixels formed on (e.g., imprinted in) or disposed on (e.g., adhered to) at least one of the layers 2011, 2012, 2013 of the eyepiece 2010. The detector pixels can be formed or disposed on at least one layer 2011, 2012, 2013, at least a portion of which is transparent and disposed at a location in front of the user's eye 2020 when the user wears the HMD such that the transparent portion transmits light from a portion of the environment 2021 in front of the user and the eyepiece 2011 to the user's eye 2020 to provide a view of the portion of the environment 2021. The sensor array 2030 can comprise wafer scale optics. The wafer scale optics can be integrated in or on the eyepiece. In various implementations, the wafer scale optics are not included in a housing that excludes the eyepiece 2010. For example, the wafer scale optics are not included in a separate housing from the housing that houses the eyepiece 2010. Rather, the wafer scale optics may be included in or on one of the layers 2011, 2012, 2013 of the eyepiece 2010. The wafer scale optics may, for example, comprise an imaging lens configured to form an image of the object on the sensor array. The wafer scale optics may therefore comprise an optical element having optical power, and the wafer scale optics may comprise a refractive lens.

In various implementations, a light source (e.g., 1032 in FIGS. 10A-10B) may be positioned such that light from the light source may be directed to the object 2020 for imaging and reflected to the sensor array 2030. In some designs, the light source may project light in the infrared (IR) or near-IR spectrum, and the sensor array 2030 may be sensitive to such light or a subset of such light. In some implementations, the light source faces forward toward the eyepiece and the environment in front of the eye such that light is reflected from the reflector to the eye. In some implementations, the light source faces toward the front of the eye such that light illuminates the eye.

In some implementations, the sensor array 2030 can be sensitive to or able to capture at least a subset (such as a non-empty subset or a subset of less than all) of the first range of wavelengths reflected by the reflector 2050. For example, the reflector 2050 may reflect IR light in a range of 700 nm to 1.5 μm, and the sensor array 2030 may be sensitive to or able to capture near IR light at wavelengths from 700 nm to 900 nm. As another example, the reflector 2050 may reflect IR light in the a range of 700 nm to 1.5 μm, and the sensor array 2030 may include a filter that filters out IR light in the range of 900 nm to 1.5 μm such that the sensor array 2030 can capture near IR light at wavelengths from 700 nm to 900 nm. However, other configurations are possible and the sensor array 2030 may be sensitive to a wider range of wavelengths, possibly more than is reflected by the reflector 2050.

In various implementations, the light from the light source may be reflected toward the sensor array 2030 by a reflector 2050. As shown in FIG. 11A, the reflector 2050 may be disposed in or on the eyepiece 2010. The reflector 2050 is shown on layer 2013, but may be formed on any one or more of the layers 2011, 2012, 2013 of the eyepiece 2010 (e.g., in or on any one or more of a waveguide, an illumination layer, a dimmer layer, a front lens, a rear lens, a cosmetic or decorative lens, etc.) or the reflector 2050 may comprise one of the layers 2011, 2012, 2013 of the eyepiece 2010. In some instances, the reflector 2050 can be disposed in or on at least one layer 2011, 2012, 2013 of the eyepiece 2010, at least a portion of which is transparent and disposed at a location in front of the user's eye 2020 when the user wears the HMD such that the transparent portion transmits light from a portion of the environment 2021 in front of the user and the eyepiece 2011 to the user's eye 2020 to provide a view of the portion of the environment 2021. In some instances, the reflector 2050 may comprise a hot mirror. In some instances, the reflector 2050 may comprise an optical coating.

The reflector 2050 may be configured to reflect or redirect light of a first range of wavelengths (e.g., IR or near-IR light) while transmitting light of a second range of wavelengths (e.g., visible light). In some such implementations, IR light 2060a from the eye 2020 propagates to and reflects from the reflector 2050, resulting in reflected IR light 2060b, which at least a portion of which can be received and imaged by the sensor array 2030.

In some implementations, the reflector 2050 may comprise a diffractive optical element (DOE), such as for example, a diffractive optical element described herein. In some designs, for example, the reflector 2050 may comprise a diffraction grating. In some instances, the reflector 2050 may be a reflective diffractive optical element that diffracts light of a first range of wavelengths (e.g., IR or near-IR light) via reflective diffraction wherein light reflected from said reflector is diffracted while transmitting light of a second range of wavelengths (e.g., visible light). In some such implementations, IR light 2060a from the eye 2020 propagates to and diffracts from the reflector 2050 (e.g., IR light reflected from the reflector is diffracted), resulting in diffracted IR light 2060b, which at least a portion of which can be received and imaged by the sensor array 2030.

In some implementations, the reflector 2050 comprises a DOE such as a Holographic Optical Element (HOE), a holographic mirror (HM), or a volumetric diffractive optical element (VDOE). In some implementations, any of these may have optical power. In some instances, the reflector 2050 comprises a diffractive optical element such as a cholesteric liquid crystal diffraction optical element or diffraction grating, which can be configured to increase and/or optimize, among other things, any one or more of polarization selectivity, bandwidth, phase profile, spatial variation of diffraction properties, spectral selectivity and/or high diffraction efficiencies. For example, any of the CLCs or CLCGs described in U.S. patent application Ser. No. 15/835,108, filed Dec. 7, 2017, entitled “Diffractive Devices Based On Cholesteric Liquid Crystal,” which is incorporated by reference herein in its entirety for all it discloses, can be implemented as a reflector 2050 or cholesteric diffractive optical element as described herein. In some embodiments, the reflector 2050 may be a switchable DOE that can be switched between “on” states in which they actively diffract, and “off” states in which they do not significantly diffract.

In some implementations, the reflector 2050 or diffractive optical element may comprise any liquid crystal gratings or structures. The above described CLCs or CLCGs may be one example of a liquid crystal grating or structure. Other liquid crystal gratings or structures may also include liquid crystal features and/or patterns that have a size less than the wavelength of visible light and may comprise what are referred to as Pancharatnam-Berry Phase Effect (PBPE) structures, metasurfaces, or metamaterials. For example, any of the PBPE structures, metasurfaces, or metamaterials described in U.S. Patent Publication No. 2017/0010466, entitled “Display System With Optical Elements For In-Coupling Multiplexed Light Streams”; U.S. patent application Ser. No. 15/879,005, filed Jan. 24, 2018, entitled “Antireflection Coatings For Metasurfaces”; or U.S. patent application Ser. No. 15/841,037, filed Dec. 13, 2017, entitled “Patterning Of Liquid Crystals Using Soft-Imprint Replication Of Surface Alignment Patterns,” each of which is incorporated by reference herein in its entirety for all it discloses, can be implemented as a reflector 2050 as described herein. Such structures may be configured to manipulate light, such as for beam steering, wavefront shaping, separating wavelengths and/or polarizations, and combining different wavelengths and/or polarizations can include liquid crystal gratings with metasurface(s), otherwise referred to as metamaterials liquid crystal gratings or liquid crystal gratings with PBPE structures. In some implementations, liquid crystal gratings with PBPE structures can combine the high diffraction efficiency and low sensitivity to angle of incidence of liquid crystal gratings with the high wavelength sensitivity of the PBPE structures.

In some implementations, a refractive or transmissive diffractive optical element 2040 can be configured to receive light 2060b reflected from the reflector 2050 and redirect at least a portion 2060c of the light toward the sensor array 2030. As illustrated in FIG. 11A, the refractive or transmissive diffractive optical element 2040 is disposed on layer 2012, but can be disposed in or on any one of the layers 2011, 2012, 2013 of the eyepiece 2010 (e.g., in or on any one or more of a waveguide, an illumination layer, a dimmer layer, a front lens, a rear lens, a cosmetic or decorative lens, etc.). In some instances, the refractive or transmissive diffractive optical element 2040 may be imprinted on one of the layers 2011, 2012, 2013 of the eyepiece 2010.

As an example, the sensor array 2030 can be disposed in or on a first layer 2011 of the eyepiece 2010 and the refractive or transmissive diffractive optical element 2040 can be disposed in or on a second different layer 2012 of the eyepiece 2010. The first 2011 and/or second 2012 layers can include a portion thereof which is transparent to transmit light therethrough.

In various implementations, the refractive or transmissive diffractive optical element 2040 can be disposed in an optical path between the reflector 2050 and the sensor array 2030. For example, the reflector 2050 can be disposed on layer 2013, the refractive or transmissive diffractive optical element 2040 can be disposed on layer 2012, and the sensor array 2030 can be disposed on layer 2011. Layer 2012 can be disposed between layer 2013 and layer 2011. The layers, 2011, 2012, and 2013 can be transparent to transmit light from a portion of the environment 2021 in front of the user.

In some instances, the refractive or transmissive diffractive optical element 2040 can comprise at least one lens aligned with respect to the sensor array 2030 such that light from the reflector 2050 can pass through at least one lens to the sensor array 2030 to form images thereon. As an example, at least one lens can comprise at least one diffractive optical element. As another example, at least one lens can comprise at least one refractive lens. The at least one lens can comprise at least one off-axis refractive lens. In various implementations, at least one lens can comprise wafer scale optics. In some implementations, at least one lens can have optical power.

In some instances, the refractive or transmissive diffractive optical element 2040 may comprise a transmissive diffractive optical element (DOE) or transmissive diffraction grating. In some implementations, the transmissive DOE or diffraction grating may comprise a Holographic Optical Element (HOE), a holographic mirror (OAHM), or a volumetric diffractive optical element (OAVDOE). In some implementations, the transmissive DOE may comprise an off-axis DOE, an off-axis diffraction grating, an off-axis Holographic Optical Element (HOE), an off-axis holographic mirror (OAHM), or an off-axis volumetric diffractive optical element (OAVDOE). In some implementations, any of these may have optical power. In some embodiments, an OAHM may have optical power as well, in which case it can be an off-axis volumetric diffractive optical element (OAVDOE). In some instances, the transmissive diffractive optical element 2040 may be a cholesteric liquid crystal diffraction optical element or diffraction grating, such as an off-axis cholesteric liquid crystal diffraction grating (OACLCG), which can be configured to increase and/or optimize, among other things, any one or more of polarization selectivity, bandwidth, phase profile, spatial variation of diffraction properties, spectral selectivity and/or high diffraction efficiencies. For example, any of the CLCs or CLCGs described in U.S. patent application Ser. No. 15/835,108, filed Dec. 7, 2017, entitled “Diffractive Devices Based On Cholesteric Liquid Crystal,” which is incorporated by reference herein in its entirety for all it discloses, can be implemented as a cholesteric diffractive optical element 2040 as described herein. In some embodiments, the transmissive diffractive optical element 2040 may be a switchable DOE that can be switched between “on” states in which they actively diffract, and “off” states in which they do not significantly diffract.

In some implementations, the transmissive diffractive optical element 2040 may be any optically transmissive liquid crystal gratings. The above described CLCs or CLCGs may be one example of a liquid crystal grating. Other liquid crystal gratings may also include liquid crystal features and/or patterns that have a size less than the wavelength of visible light and may comprise what are referred to as Pancharatnam-Berry Phase Effect (PBPE) structures, metasurfaces, or metamaterials. For example, any of the PBPE structures, metasurfaces, or metamaterials described in U.S. Patent Publication No. 2017/0010466, entitled “Display System With Optical Elements For In-Coupling Multiplexed Light Streams”; U.S. patent application Ser. No. 15/879,005, filed Jan. 24, 2018, entitled “Antireflection Coatings For Metasurfaces”; or U.S. patent application Ser. No. 15/841,037, filed Dec. 13, 2017, entitled “Patterning Of Liquid Crystals Using Soft-Imprint Replication Of Surface Alignment Patterns,” each of which is incorporated by reference herein in its entirety for all it discloses, can be implemented as an off-axis optical element 2040 as described herein. Such structures may be configured to manipulate light, such as for beam steering, wavefront shaping, separating wavelengths and/or polarizations, and combining different wavelengths and/or polarizations can include liquid crystal gratings with metasurface, otherwise referred to as metamaterials liquid crystal gratings or liquid crystal gratings with PBPE structures. In some implementations, liquid crystal gratings with PBPE structures can combine the high diffraction efficiency and low sensitivity to angle of incidence of liquid crystal gratings with the high wavelength sensitivity of the PBPE structures.

As described herein, the imaging system 2000 can be used in head mounted displays to track the gaze of the user based on images of the at least one of: the eye of the user, the part of the eye, or the portion of tissue surrounding the eye. For example, as illustrated in FIG. 11A, the sensor array 2030 can be a forward facing camera configured to image at least part of the object (e.g., the eye 2020 of the user, a part of the eye 2020, or a portion of tissue surrounding the eye 2020) based at least in part on light received from the reflector 2050, which is disposed forward of sensor array 2030. At least part of the object 2020 can be disposed rearward of the sensor array 2030.

In some implementations, the imaging system 2000 can be used to image objects in the environment 2021 in front of the eyepiece 2020. For example, as illustrated in FIG. 11B, the sensor array 2030 can be a backward facing camera configured to image at least part of the object 2022 based at least in part on light received from the reflector 2050, which is disposed rearward of the sensor array 2030. At least part of the object 2022 can be disposed forward of the sensor array 2030 in the environment 2021 in front of the user. In the illustrated configuration, the refractive or transmissive diffractive optical element is disposed between the reflector 2050 and the sensor array 2030.

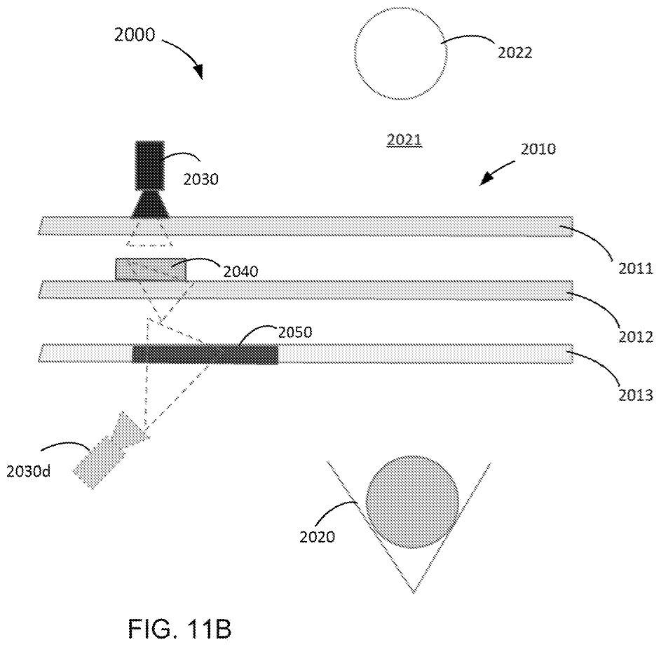

As disclosed herein, as shown in FIG. 11A, the sensor array 2030 can be disposed in or on a first layer 2011 of the eyepiece 2010 and the refractive or transmissive diffractive optical element 2040 can be disposed in or on a second different layer 2012 of the eyepiece 2010. In various implementations, the refractive or transmissive diffractive optical element 2040 can include at least one imaging lens aligned with the sensor array 2030 to form images on the sensor array 2030. In some implementations, the refractive or transmissive comprises an off-axis optical element such as an off-axis lens. In some instances, the refractive or transmissive diffractive optical element 2040 can be disposed on the same layer as the sensor array 2030. For example, as shown in FIG. 12A, the sensor array 2030 can be disposed in or on a first side 2011a of a layer 2011 of the eyepiece 2010 and the refractive or transmissive diffractive optical element 2040a can be disposed in or on a second opposite side 2011b of the layer 2011. In various implementations, the refractive or transmissive diffractive optical element 2040a can include at least one imaging lens aligned with the sensor array 2030 to form images on the sensor array 2030. In some designs, the refractive or transmissive diffractive optical element comprises an off-axis optical element such as an off-axis lens. In some implementations, the layer 2011 on which the sensor array 2030 and the refractive or transmissive diffractive optical element 2040a are disposed in or on can include a portion thereof which is transparent to transmit light.

In some implementations, the eyepiece 2010 can include more than one refractive or transmissive diffractive optical elements 2040. The refractive or transmissive diffractive optical elements 2040 can be on the same and/or different layers 2011, 2012, 2013 of the eyepiece 2010 from the sensor array 2030. For example, as shown in FIG. 12B, the eyepiece 2010 includes refractive or transmissive diffractive optical elements 2040a, 2040b on both a same layer 2011 and a different layer 2012 of the eyepiece 2010 from the sensor array 2030. In various implementations, the refractive or transmissive diffractive optical elements 2040a, 2040b can include imaging lenses aligned with the sensor array 2030 to form images on the sensor array 2030. The layers 2011, 2012 on which the sensor array 2030 and the refractive or transmissive diffractive optical elements 2040a, 2040b are disposed in or on can each include a portion thereof which is transparent to transmit light. In some implementations, the refractive or transmissive diffractive optical elements may include an off-axis optical element to redirect the beam of light and an imaging lens to form an image on the sensor array 2030.

In some instances, multiple refractive or transmissive diffractive optical elements 2040a, 2040b can be on one or more layers different from the sensor array 2030. For example, the refractive or transmissive diffractive optical elements 2040a, 2040b can be on opposite sides 2012a, 2012b of the same layer 2012, but different from the layer 2011 from the sensor array 2030. As another example, the refractive or transmissive diffractive optical elements 2040a, 2040b can be on separate layers (e.g., 2012, 2013 of FIGS. 11A-11B) from each other and from the sensor array 2030. The refractive or transmissive diffractive optical elements 2040a, 2040b can include one or more lenses (e.g., a single lens or multiple lenses). In some instances, the one or more lenses can be used with the sensor array 2030 to form images thereon. In some designs the refractive or transmissive diffractive may comprise at least one refractive or transmissive diffractive configured to redirect the beam of light and at least one imaging lens on same or different layers.

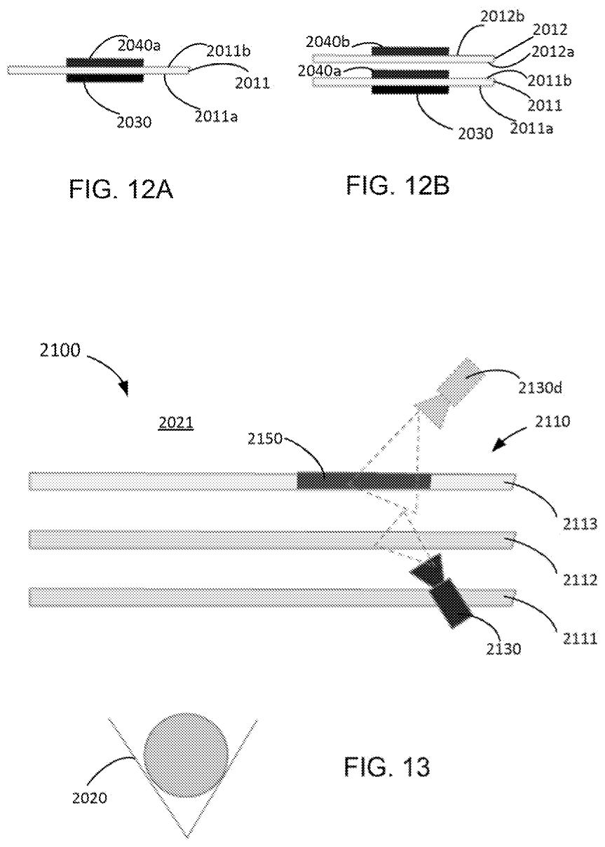

FIG. 13 schematically illustrates an example implementation of another imaging system 2100. In various implementations, the eyepiece 2110 can be similar to the eyepiece 2010 described with respect to FIGS. 11A-11B. For example, the eyepiece 2010 can include one or more layers 2111, 2112, 2113. The layers 2111, 2112, 2113 can be similar to layers 2011, 2012 2013 described with respect to FIGS. 11A-11B (e.g., one or more of a waveguide, an illumination layer, a dimmer layer, a front lens, a rear lens, a cosmetic or decorative lens, etc.). The one or more layers 2111, 2112, 2113 can be planar. The imaging system 2100 can also include a reflector 2150 similar to reflector 2050 described with respect to FIGS. 11A-11B. The imaging system 2100 can also include an imager 2130. The imager 2130 can include a sensor array (similar to sensor array 2030 described with respect to FIGS. 11A-11B) and wafer scale imaging optics. For example, the sensor array and wafer scale imaging optics of the imager 2130 can be integrated in or on the eyepiece 2110. In various implementations, the wafer scale imaging optics integrated in or on the eyepiece 2110 is not included in a housing that excludes the eyepiece 2110. For example, the camera does not necessarily comprise a sensor array including wafer scale optics in a housing that is disposed on the eyepiece, which may also be included in a housing. Rather, the wafer scale optics is included on a layer of the eye piece and aligned with the sensor array, which may be included on the same or a different layer of the eyepiece, which may be included in a housing.

The imaging system 2100 can be similar to the imaging system 2000 described with respect to FIGS. 11A-11B. In some such designs, the imaging system 2100 may or may not include a refractive or transmissive diffractive optical element 2040 comprising an off-axis optical element as discussed above in connection with FIGS. 11A-11B. In some instances, the wafer scale imaging optics may include one or more imaging lenses (e.g., can be similar to optical elements 2040a, 2040b described with respect to FIGS. 12A-12B) disposed on the same or different layer from the sensor array. The imaging lens can be aligned with respect to the sensor array such that light from the reflector 2150 can pass through to the sensor array to form images thereon.

### Example Routine to Image an Object

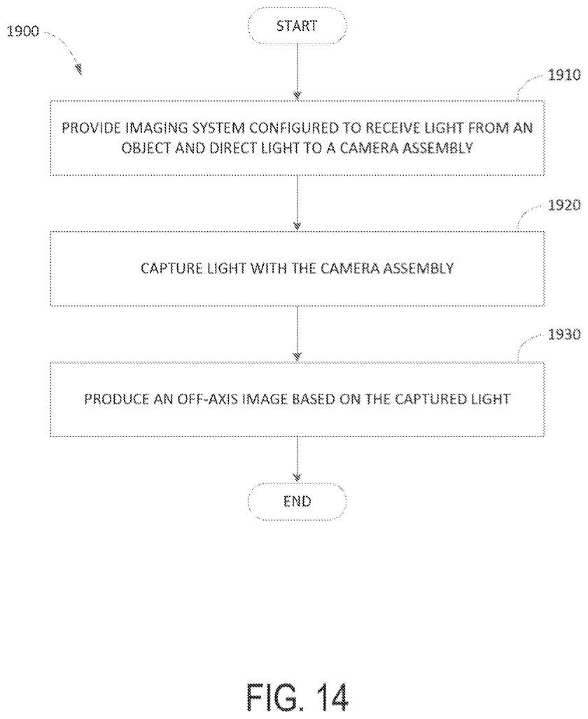

FIG. 14 is a process flow diagram of an illustrative routine to image an object (e.g., an eye of the user or an object in the environment) using an off-axis camera (or sensor array), e.g., camera assembly 630 of FIG. 6, camera assembly 1030 of FIG. 10A, sensor array 2030 of FIGS. 11A, 11B, or imager 2130 of FIG. 13. The routine 1900 describes how light from an object can be directed to a camera assembly that is positioned away from or off to the side relative to the object (e.g., the eye) to image the object as though the camera assembly was pointed directly toward the object from straight in front of the object.

At block 1910, an imaging system is provided that is configured to receive light from the object and direct the light to a camera assembly. The imaging system may be one or more of the imaging systems as described above in connection to FIGS. 10A, 10B, 11A, 11B, and 13.

At block 1920, the light is captured with a camera assembly (e.g., camera assembly 630 of FIG. 6, camera assembly 1030 of FIGS. 10A-10B, sensor array 2030 of FIGS. 11A, 11B, or imager 2130 of FIG. 13). The camera assembly may be disposed of to the side of the eye or the pupil of the eye or the straightforward line of site of the eye. The camera assembly may be disposed of the side (e.g., temporal) of the eyepiece. The camera assembly may be a camera in a forward facing or backward facing configuration. At block 1930, an image of the object may be produced based on the captured light, as described herein and throughout this disclosure.

In some embodiments, the routine 1900 may include an optional step (not shown) of illuminating the object with light from a light source (e.g., light source of FIG. 6 or light source 1032 of FIGS. 10A-10B). In some embodiments, the light may comprise range of wavelengths including IR light.

In some embodiments, the image produced at block 1930 may be processed and analyzed, for example, using image-processing techniques. In some implementations, the analyzed image may be used to perform one or more of: eye tracking; biometric identification; multiscopic reconstruction of a shape of an eye; estimating an accommodation state of an eye; or imaging a retina, iris, other distinguishing pattern of an eye, and evaluate a physiological state of the user based, in part, on the analyzed off-axis image, as described above and throughout this disclosure. In some implementations, the analyzed image may be used to track an object in the environment.

In various embodiments, the routine 1900 may be performed by a hardware processor (e.g., the local processing and data module 140 of FIG. 2) configured to execute instructions stored in a memory. In other embodiments, a remote computing device (in network communication with the display apparatus) with computer-executable instructions can cause the display apparatus to perform aspects of the routine 1900.

### Examples

The disclosure provides various examples of head mounted displays. Such examples include but are not limited to the following examples.

Part I

1. A head mounted display comprising:
a frame configured to be supported on the head of the user;
an eyepiece coupled to said frame and configured to be disposed in front of an eye of the user, said eyepiece comprising a plurality of layers;
an image injection device configured to provide image content to the eyepiece for viewing by the user;
a sensor array integrated in or on the eyepiece;
a reflector disposed in or on the eyepiece, the reflector configured to reflect light received from an object for imaging by said sensor array; and
a transmissive diffractive optical element disposed in or on the eyepiece, the transmissive diffractive optical element configured to receive light reflected from the reflector and transmit and diffract at least a portion of the light toward the sensor array.

2. The head mounted display of Example 1, wherein the said eyepiece is configured to direct light from said image injection device to said user's eye to present image content to said user.

3. The head mounted display of any of the examples above, wherein the plurality of layers comprises one or more waveguides configured to receive light from said image injection device and guide at least a portion of the light therein by total internal reflection to provide image content to the user.

4. The head mounted display of any of the examples above, wherein the plurality of layers comprises a plurality of waveguides, different waveguides arranged to provide different color image content.

5. The head mounted display of any of the examples above, wherein the plurality of layers comprises a plurality of waveguides, different waveguides or groups of waveguides configured to project light into the user's eye to display image content with different amounts of divergences as if projected from different distances from the user's eye.

6. The head mounted display of any of the examples above, wherein the plurality of layers comprises one or more of a decorative or cosmetic lens, a front lens, a dimmer, a rear lens, an illumination layer, or a prescription lens configured to provide for refractive correction for a user having a refractive deficiency.

7. The head mounted display of any of the examples above, wherein said eyepiece comprises at least a portion that is transparent and disposed at a location in front of the user's eye when the user wears the head mounted display device such that the transparent portion transmits light from a portion of the environment in front of the user and the eyepiece to the user's eye to provide a view of the portion of the environment in front of the user and the eyepiece.

8. The head mounted display of any of the examples above, wherein said plurality of layers comprises at least one glass or plastic layer that is transparent.

9. The head mounted display of any of the examples above, wherein said sensor array comprises a plurality of detector pixels formed on at least one of said layers.

10. The head mounted display of any of the examples above, wherein said sensor array comprises a plurality of detector pixels disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

11. The head mounted display of any of the examples above, wherein said sensor array comprises wafer scale optics.

12. The head mounted display of any of the examples above, wherein said transmissive diffractive optical element comprises at least one diffractive lens aligned with respect to said sensor array such that light from said reflector passes through said at least one diffractive lens to said sensor array to form images thereon.

13. The head mounted display of Example 12, wherein said sensor array is disposed in or on a first layer of said plurality of layers of said eyepiece and said at least one diffractive lens is disposed in or on a second different layer of said plurality of layers of said eyepiece.

14. The head mounted display of Example 13, wherein first and second layers each include at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

15. The head mounted display of Example 12, wherein said sensor array is disposed in or on a first side of a layer of said plurality of layers of said eyepiece and said at least one diffractive lens is disposed in or on a second opposite side of said layer.

16. The head mounted display of Example 15, wherein said layer on which said sensor array and at least one lens is disposed in or on includes at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

17. The head mounted display of any of the examples above, wherein said reflector comprises a hot mirror.

18. The head mounted display of any of the examples above, wherein said reflector is configured to reflect light of a first range of infrared (IR) or near-IR wavelengths while transmitting light of a second range of visible wavelengths.

19. The head mounted display of any of the examples above, wherein said reflector is formed on at least one of said plurality of layers.

20. The head mounted display of any of the examples above, wherein said reflector comprises one of said plurality of layers.

21. The head mounted display of any of the examples above, wherein said reflector comprises an optical coating.

22. The head mounted display of any of the examples above, wherein said reflector is disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

23. The head mounted display of any of the examples above, wherein said transmissive diffractive optical element is disposed in an optical path between said reflector and said sensor array.

24. The head mounted display of any of the examples above, wherein said reflector is disposed on a first layer of said plurality of layers, said transmissive diffractive optical element is disposed a second layer of said plurality of layers, and said sensor array is disposed on a third layer of said plurality of layers, said second layer disposed between said first and third layers.

25. The head mounted display of Example 24, wherein said at least a portion of said first, second, and third layers are transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

26. The head mounted display of any of the examples above, wherein said transmissive diffractive optical element comprises a transmissive diffraction grating.

27. The head mounted display of any of the examples above, wherein said transmissive diffractive optical element comprises a transmissive holographic optical element, a transmissive volumetric diffractive optical element (OAVDOE), or a transmissive cholesteric liquid crystal diffraction grating (OACLCG).

28. The head mounted display of any of the examples above, wherein said transmissive diffractive optical element has optical power.

29. The head mounted display of any of any of the examples above, wherein at least a portion of said transmissive diffractive optical element is imprinted on one of the layers of the plurality of layers of the eyepiece.

30. The head mounted display of any of the examples above, wherein the sensor array is a forward facing camera configured to image at least part of the object based at least in part on light received from said reflector, which is disposed forward of said sensor array, the at least part of the object being disposed rearward of said sensor array and comprising at least one of: the eye of the user, a part of the eye, or a portion of tissue surrounding the eye.

31. The head mounted display of any of the examples above, further comprising a light source emitting light of a first range of wavelengths toward at least one of: the eye of the user, a part of the eye, or a portion of tissue surrounding the eye.

32. The head mounted display of Example 31, wherein the first range of wavelengths comprises light in at least one of the infrared (IR) or near-IR spectrum.

33. The head mounted display of any of the examples above, wherein the head mounted display is configured to track the gaze of the user based on images of the at least one of: the eye of the user, the part of the eye, or the portion of tissue surrounding the eye.

34. The head mounted display of any of examples above, wherein the sensor array is further configured to image an object in the environment in front of said eyepiece.

35. The head mounted display of Example 34, wherein the sensor array is a backward facing camera configured to image at least part of the object based at least in part on light received from said reflector, which is rearward of said sensor array, the at least part of the object disposed forward of the sensor array in the environment in front of the user.

36. The head mounted display of any of examples above, wherein head mounted display comprises eyewear.

Part II

1. A head mounted display comprising:
a frame configured to be supported on the head of the user;
an eyepiece coupled to said frame and configured to be disposed in front of an eye of the user, said eyepiece comprising a plurality of layers;
an image injection device configured to provide image content to the eyepiece for viewing by the user;
a sensor array integrated in or on the eyepiece;
a reflector disposed in or on the eyepiece, the reflector configured to reflect light received from an object for imaging by said sensor array; and
a transmissive refractive optical element disposed in or on the eyepiece, the transmissive refractive optical element configured to receive light reflected from the reflector and refract at least a portion of the light toward the sensor array.

2. The head mounted display of Example 1, wherein the said eyepiece is configured to direct light from said image injection device to said user's eye to present image content to said user.

3. The head mounted display of any of the examples above, wherein the plurality of layers comprises one or more waveguides configured to receive light from said image injection device and guide at least a portion of the light therein by total internal reflection to provide image content to the user.

4. The head mounted display of any of the examples above, wherein the plurality of layers comprises a plurality of waveguides, different waveguides arranged to provide different color image content.

5. The head mounted display of any of the examples above, wherein the plurality of layers comprises a plurality of waveguides, different waveguides or groups of waveguides configured to project light into the user's eye to display image content with different amounts of divergences as if projected from different distances from the user's eye.

6. The head mounted display of any of the examples above, wherein the plurality of layers comprises one or more of a decorative or cosmetic lens, a front lens, a dimmer, a rear lens, an illumination layer, or a prescription lens configured to provide for refractive correction for a user having a refractive deficiency.

7. The head mounted display of any of the examples above, wherein said eyepiece comprises at least a portion that is transparent and disposed at a location in front of the user's eye when the user wears the head mounted display device such that the transparent portion transmits light from a portion of the environment in front of the user and the eyepiece to the user's eye to provide a view of the portion of the environment in front of the user and the eyepiece.

8. The head mounted display of any of the examples above, wherein said plurality of layers comprises at least one glass or plastic layer that is transparent.

9. The head mounted display of any of the examples above, wherein said sensor array comprises a plurality of detector pixels formed on at least one of said layers.

10. The head mounted display of any of the examples above, wherein said sensor array comprises a plurality of detector pixels disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

11. The head mounted display of any of the examples above, wherein said sensor array comprises wafer scale optics.

12. The head mounted display of any of the examples above, wherein said refractive optical element comprises at least one lens aligned with respect to said sensor array such that light from said reflector passes through said at least one lens to said sensor array to form images thereon.

13. The head mounted display of Example 12, wherein said sensor array is disposed in or on a first layer of said plurality of layers of said eyepiece and said at least one lens is disposed in or on a second different layer of said plurality of layers of said eyepiece.

14. The head mounted display of Example 13, wherein first and second layers each include at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

15. The head mounted display of Example 12, wherein said sensor array is disposed in or on a first side of a layer of said plurality of layers of said eyepiece and said at least one lens is disposed in or on a second opposite side of said layer.

16. The head mounted display of Example 15, wherein said layer on which said sensor array and at least one lens is disposed in or on includes at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

17. The head mounted display of any of Examples 12-16, wherein at least one lens comprises a diffractive optical element.

18. The head mounted display of any of Examples 12-16, wherein at least one lens comprises at least one refractive lens.

19. The head mounted display of any of Examples 12-16, wherein at least one lens comprises wafer scale optics.

20. The head mounted display of any of the examples above, wherein said reflector comprises a hot mirror.

21. The head mounted display of any of the examples above, wherein said reflector is configured to reflect light of a first range of infrared (IR) or near-IR wavelengths while transmitting light of a second range of visible wavelengths.

22. The head mounted display of any of the examples above, wherein said reflector is formed on at least one of said plurality of layers.

23. The head mounted display of any of the examples above, wherein said reflector comprises one of said plurality of layers.

24. The head mounted display of any of the examples above, wherein said reflector comprises an optical coating.

25. The head mounted display of any of the examples above, wherein said reflector is disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

26. The head mounted display of any of the examples above, wherein said refractive optical element is disposed in an optical path between said reflector and said sensor array.

27. The head mounted display of any of the examples above, wherein said reflector is disposed on a first layer of said plurality of layers, said refractive optical element is disposed on a second layer of said plurality of layers, and said sensor array is disposed on a third layer of said plurality of layers, said second layer disposed between said first and third layers.

28. The head mounted display of Example 27, wherein said at least a portion of said first, second, and third layers are transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

29. The head mounted display of any of the examples above, wherein said refractive optical element comprise an off-axis optical element.

30. The head mounted display of any of the examples above, wherein said refractive optical element has optical power.

31. The head mounted display of any of the examples above, wherein said refractive optical element comprises an off-axis lens.

32. The head mounted display of any of any of the examples above, wherein at least a portion of said refractive optical element is imprinted on one of the layers of the plurality of layers of the eyepiece.

33. The head mounted display of any of the examples above, wherein the sensor array is a forward facing camera configured to image at least part of the object based at least in part on light received from said reflector, which is disposed forward of said sensor array, the at least part of the object being disposed rearward of said sensor array and comprising at least one of: the eye of the user, a part of the eye, or a portion of tissue surrounding the eye.

34. The head mounted display of any of the examples above, further comprising a light source emitting light of a first range of wavelengths toward at least one of: the eye of the user, a part of the eye, or a portion of tissue surrounding the eye.

35. The head mounted display of Example Error! Reference source not found., wherein the first range of wavelengths comprises light in at least one of the infrared (IR) or near-IR spectrum.

36. The head mounted display of any of the examples above, wherein the head mounted display is configured to track the gaze of the user based on images of the at least one of: the eye of the user, the part of the eye, or the portion of tissue surrounding the eye.

37. The head mounted display of any of examples above, wherein the sensor array is further configured to image an object in the environment in front of said eyepiece.

38. The head mounted display of Example 37, wherein the sensor array is a backward facing camera configured to image at least part of the object based at least in part on light received from said reflector, which is rearward of said sensor array, the at least part of the object disposed forward of the sensor array in the environment in front of the user.

39. The head mounted display of any of examples above, wherein head mounted display comprises eyewear.

Part III

1. A head mounted display comprising:
a frame configured to be supported on the head of the user;
an eyepiece coupled to said frame and configured to be disposed in front of an eye of the user, said eyepiece comprising a plurality of layers;
an image injection device configured to provide image content to the eyepiece for viewing by the user;
a sensor array integrated in or on the eyepiece;
a reflector disposed in or on the eyepiece, the reflector configured to reflect light received from an object for imaging by said sensor array; and
an off-axis optical element disposed in or on the eyepiece, the off-axis optical element configured to receive light reflected from the reflector and direct at least a portion of the light toward the sensor array.

2. The head mounted display of Example 1, wherein the said eyepiece is configured to direct light from said image injection device to said user's eye to present image content to said user.

3. The head mounted display of any of the examples above, wherein the plurality of layers comprises one or more waveguides configured to receive light from said image injection device and guide at least a portion of the light therein by total internal reflection to provide image content to the user.

4. The head mounted display of any of the examples above, wherein the plurality of layers comprises a plurality of waveguides, different waveguides arranged to provide different color image content.

5. The head mounted display of any of the examples above, wherein the plurality of layers comprises a plurality of waveguides, different waveguides or groups of waveguides configured to project light into the user's eye to display image content with different amounts of divergences as if projected from different distances from the user's eye.

6. The head mounted display of any of the examples above, wherein the plurality of layers comprises one or more of a decorative or cosmetic lens, a front lens, a dimmer, a rear lens, an illumination layer, or a prescription lens configured to provide for refractive correction for a user having a refractive deficiency.

7. The head mounted display of any of the examples above, wherein said eyepiece comprises at least a portion that is transparent and disposed at a location in front of the user's eye when the user wears the head mounted display device such that the transparent portion transmits light from a portion of the environment in front of the user and the eyepiece to the user's eye to provide a view of the portion of the environment in front of the user and the eyepiece.

8. The head mounted display of any of the examples above, wherein said plurality of layers comprises at least one glass or plastic layer that is transparent.

9. The head mounted display of any of the examples above, wherein said sensor array comprises a plurality of detector pixels formed on at least one of said layers.

10. The head mounted display of any of the examples above, wherein said sensor array comprises a plurality of detector pixels disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

11. The head mounted display of any of the examples above, wherein said sensor array comprises wafer scale optics.

12. The head mounted display of any of the examples above, wherein said off-axis optical element comprises at least one lens aligned with respect to said sensor array such that light from said reflector passes through said at least one lens to said sensor array to form images thereon.

13. The head mounted display of Example 12, wherein said sensor array is disposed in or on a first layer of said plurality of layers of said eyepiece and said at least one lens is disposed in or on a second different layer of said plurality of layers of said eyepiece.

14. The head mounted display of Example 13, wherein first and second layers each include at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

15. The head mounted display of Example 12, wherein said sensor array is disposed in or on a first side of a layer of said plurality of layers of said eyepiece and said at least one lens is disposed in or on a second opposite side of said layer.

16. The head mounted display of Example 15, wherein said layer on which said sensor array and at least one lens is disposed in or on includes at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

17. The head mounted display of any of Examples 12-16, wherein at least one lens comprises a diffractive optical element.

18. The head mounted display of any of Examples 12-16, wherein at least one lens comprises at least one refractive lens.

19. The head mounted display of any of Examples 12-16, wherein at least one lens comprises wafer scale optics.

20. The head mounted display of any of the examples above, wherein said reflector comprises a hot mirror.

21. The head mounted display of any of the examples above, wherein said reflector is configured to reflect light of a first range of infrared (IR) or near-IR wavelengths while transmitting light of a second range of visible wavelengths.

22. The head mounted display of any of the examples above, wherein said reflector is formed on at least one of said plurality of layers.

23. The head mounted display of any of the examples above, wherein said reflector comprises one of said plurality of layers.

24. The head mounted display of any of the examples above, wherein said reflector comprises an optical coating.

25. The head mounted display of any of the examples above, wherein said reflector is disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

26. The head mounted display of any of the examples above, wherein said off-axis optical element is disposed in an optical path between said reflector and said sensor array.

27. The head mounted display of any of the examples above, wherein said reflector is disposed on a first layer of said plurality of layers, said off-axis optical element is disposed a second layer of said plurality of layers, and said sensor array is disposed on a third layer of said plurality of layers, said second layer disposed between said first and third layers.

28. The head mounted display of Example 27, wherein said at least a portion of said first, second, and third layers are transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

29. The head mounted display of any of the examples above, wherein said off-axis optical element comprises a diffractive optical element (DOE) or diffraction grating.

30. The head mounted display of any of the examples above, wherein said off-axis optical element comprises an off-axis diffractive optical element (DOE), an off-axis diffraction grating, an off-axis holographic mirror (OAHM), an off-axis volumetric diffractive optical element (OAVDOE), or an off-axis cholesteric liquid crystal diffraction grating (OACLCG).

31. The head mounted display of any of the examples above, wherein said off-axis optical element has optical power.

32. The head mounted display of any of any of the examples above, wherein at least a portion of said off-axis optical element is imprinted on one of the layers of the plurality of layers of the eyepiece.

33. The head mounted display of any of the examples above, wherein the sensor array is a forward facing camera configured to image at least part of the object based at least in part on light received from said reflector, which is disposed forward of said sensor array, the at least part of the object being disposed rearward of said sensor array and comprising at least one of: the eye of the user, a part of the eye, or a portion of tissue surrounding the eye.

34. The head mounted display of any of the examples above, further comprising a light source emitting light of a first range of wavelengths toward at least one of: the eye of the user, a part of the eye, or a portion of tissue surrounding the eye.

35. The head mounted display of Example Error! Reference source not found., wherein the first range of wavelengths comprises light in at least one of the infrared (IR) or near-IR spectrum.

36. The head mounted display of any of the examples above, wherein the head mounted display is configured to track the gaze of the user based on images of the at least one of: the eye of the user, the part of the eye, or the portion of tissue surrounding the eye.

37. The head mounted display of any of examples above, wherein the sensor array is further configured to image an object in the environment in front of said eyepiece.

38. The head mounted display of Example 37, wherein the sensor array is a backward facing camera configured to image at least part of the object based at least in part on light received from said reflector, which is rearward of said sensor array, the at least part of the object disposed forward of the sensor array in the environment in front of the user.

39. The head mounted display of any of examples above, wherein head mounted display comprises eyewear.

40. A head mounted display comprising:
a frame configured to be supported on the head of the user;
an eyepiece coupled to said frame and configured to be disposed in front of an eye of the user, said eyepiece comprising a plurality of layers;
an image injection device configured to provide image content to the eyepiece for viewing by the user;
a sensor array integrated in or on a first layer of said plurality of layers of said eyepiece; and
at least one imaging lens aligned with said sensor array to form images on said sensor array, said at least one imaging lens disposed in or on a second layer of said plurality of layers of said eyepiece.

41. The head mounted display of Example 40, wherein the said eyepiece is configured to direct light from said image injection device to said user's eye to present image content to said user.

42. The head mounted display of any of the Examples 40-41, wherein the plurality of layers comprises one or more waveguides configured to receive light from said image injection device and guide at least a portion of the light therein by total internal reflection to provide image content to the user.

43. The head mounted display of any of the Examples 40-42, wherein the plurality of layers comprises a plurality of waveguides, different waveguides arranged to provide different color image content.

44. The head mounted display of any of the Examples 40-43, wherein the plurality of layers comprises a plurality of waveguides, different waveguides or groups of waveguides configured to project light into the user's eye to display image content with different amounts of divergences as if projected from different distances from the user's eye.

45. The head mounted display of any of the Examples 40-44, wherein the plurality of layers comprises one or more of a cosmetic window, a front lens, a dimmer, a rear lens, an illumination layer, or a prescription lens configured to provide for refractive correction for a user having a refractive deficiency.

46. The head mounted display of any of the Examples 40-45, wherein said eyepiece comprises at least a portion that is transparent and disposed at a location in front of the user's eye when the user wears the head mounted display device such that the transparent portion transmits light from a portion of the environment in front of the user and the eyepiece to the user's eye to provide a view of the portion of the environment in front of the user and the eyepiece.

47. The head mounted display of any of the Examples 40-46, wherein said plurality of layers comprises at least one glass or plastic layer that is transparent.

48. The head mounted display of any of the Examples 40-47, wherein said sensor array comprises a plurality of detector pixels formed on at least one of said layers.

49. The head mounted display of any of the Examples 40-48, wherein said sensor array comprises a plurality of detector pixels disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

50. The head mounted display of any of the Examples 40-49, further comprising a reflector and said at least one lens is aligned with respect to said sensor array such that light from said reflector passes through said at least one lens to said sensor array to form images thereon.

51. The head mounted display of any of the Examples 40-50, wherein first and second layers each include at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

52. The head mounted display of any of the Examples 40-51, wherein said at least one lens comprises a diffractive optical element.

53. The head mounted display of any of the Examples 40-51, wherein said at least one lens comprises at least one refractive lens.

54. The head mounted display of any of the Examples 40-53, wherein said at least one lens comprises wafer scale optics.

55. The head mounted display of any of the Examples 40-54, further comprising a reflector disposed in or on the eyepiece, the reflector configured to reflect light received from an object for imaging by said sensor array;

56. The head mounted display of Example 55, wherein said reflector comprises a hot mirror.

57. The head mounted display of Example 55 or 56, wherein said reflector is configured to reflect light of a first range of infrared (IR) or near-IR wavelengths while transmitting light of a second range of visible wavelengths.

58. The head mounted display of any of Examples 55-57, wherein said reflector is formed on at least one of said plurality of layers.

59. The head mounted display of any of Examples 55-58, wherein said reflector comprises one of said plurality of layers.

60. The head mounted display of any of Examples 55-59, wherein said reflector comprises an optical coating.

61. The head mounted display of any of Examples 55-60, wherein said reflector is disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

62. The head mounted display of any of the Examples 50-61, further comprising an off-axis optical element disposed in or on the eyepiece, the off-axis optical element is configured to receive light reflected from the reflector and direct at least of portion of the light toward the at least one imaging lens.

63. The head mounted display of Example 62, wherein said off-axis optical element is disposed in an optical path between said reflector and said at least one imaging lens.

64. The head mounted display of any of Examples 50-63, wherein said reflector is disposed on a third layer of said plurality of layers, said second layer disposed between said first and third layers.

65. The head mounted display of Example 64, wherein said at least a portion of said first, second, and third layers are transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

66. The head mounted display of any of Examples 62-65, wherein said off-axis optical element comprises a diffractive optical element (DOE) or diffraction grating.

67. The head mounted display of any of the Examples 62-66, wherein said off-axis optical element comprises an off-axis diffractive optical element (DOE), an off-axis diffraction grating, an off-axis holographic mirror (OAHM), an off-axis volumetric diffractive optical element (OAVDOE), or an off-axis cholesteric liquid crystal diffraction grating (OACLCG).

68. The head mounted display of any of the Examples 62-67, wherein said off-axis optical element has optical power.

69. The head mounted display of any of any of the Examples 62-68, wherein at least a portion of said off-axis optical element is imprinted on one of the layers of the plurality of layers of the eyepiece.

70. The head mounted display of any of the Examples 50-69, wherein the sensor array is a forward facing camera configured to image at least part of the object based at least in part on light received from a reflector, which is disposed forward of said sensor array, the at least part of the object being disposed rearward of said sensor array and comprising at least one of: the eye of the user, a part of the eye, or a portion of tissue surrounding the eye.

71. The head mounted display of any of the Examples 40-70, further comprising a light source emitting light of a first range of wavelengths toward at least one of: the eye of the user, a part of the eye, or a portion of tissue surrounding the eye.

72. The head mounted display of Example 71, wherein the first range of wavelengths comprises light in at least one of the infrared (IR) or near-IR spectrum.

73. The head mounted display of any of the Examples 40-72, wherein the head mounted display is configured to track the gaze of the user based on images of the at least one of: the eye of the user, the part of the eye, or the portion of tissue surrounding the eye.

74. The head mounted display of any of the Examples 40-69, wherein the sensor array is configured to image an object in the environment in front of said eyepiece.

75. The head mounted display of any of Examples 50-69 and 74, wherein the sensory array is a backward facing camera configured to image at least part of the object based at least in part on light received from a reflector, which is rearward of said sensor array, the at least part of the object disposed forward of the sensor array in the environment in front of the user.

76. The head mounted display of any of Examples 40-75, wherein head mounted display comprises eyewear.

77. A head mounted display comprising:
a frame configured to be supported on the head of the user;
an eyepiece coupled to said frame and configured to be disposed in front of an eye of the user, said eyepiece comprising a plurality of layers;
an image injection device configured to provide image content to the eyepiece for viewing by the user;
a sensor array integrated in or on a first side of a layer of said plurality of layers of said eyepiece; and
at least one imaging lens aligned with said sensor array to form images on said sensor array, said at least one imaging lens disposed in or on a second side of said layer of said plurality of layers of said eyepiece on which said sensor array is disposed.

78. The head mounted display of Example 77, wherein the said eyepiece is configured to direct light from said image injection device to said user's eye to present image content to said user.

79. The head mounted display of any of the Examples 77-78, wherein the plurality of layers comprises one or more waveguides configured to receive light from said image injection device and guide at least a portion of the light therein by total internal reflection to provide image content to the user.

80. The head mounted display of any of the Examples 77-79, wherein the plurality of layers comprises a plurality of waveguides, different waveguides arranged to provide different color image content.

81. The head mounted display of any of the Examples 77-80, wherein the plurality of layers comprises a plurality of waveguides, different waveguides or groups of waveguides configured to project light into the user's eye to display image content with different amounts of divergences as if projected from different distances from the user's eye.

82. The head mounted display of any of the Examples 77-81, wherein the plurality of layers comprises one or more of a cosmetic window, a front lens, a dimmer, a rear lens, an illumination layer, or a prescription lens configured to provide for refractive correction for a user having a refractive deficiency.

83. The head mounted display of any of the Examples 77-82, wherein said eyepiece comprises at least a portion that is transparent and disposed at a location in front of the user's eye when the user wears the head mounted display device such that the transparent portion transmits light from a portion of the environment in front of the user and the eyepiece to the user's eye to provide a view of the portion of the environment in front of the user and the eyepiece.

84. The head mounted display of any of the Examples 77-83, wherein said plurality of layers comprises at least one glass or plastic layer that is transparent.

85. The head mounted display of any of the Examples 77-84, wherein said sensor array comprises a plurality of detector pixels formed on at least one of said layers.

86. The head mounted display of any of the Examples 77-85, wherein said sensor array comprises a plurality of detector pixels disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

87. The head mounted display of any of the Examples 77-86, further comprising a reflector and said at least one lens is aligned with respect to said sensor array such that light from said reflector passes through said at least one lens to said sensor array to form images thereon.

88. The head mounted display of any of Examples 77-87, wherein said layer on which said sensor array and at least one lens is disposed in or on includes at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

89. The head mounted display of any of the Examples 77-88, wherein said at least one lens comprises a diffractive optical element.

90. The head mounted display of any of the Examples 77-88, wherein said at least one lens comprises at least one refractive lens.

91. The head mounted display of any of the Examples 77-90, wherein said at least one lens comprises wafer scale optics.

92. The head mounted display of any of the Examples 77-91, further comprising a reflector disposed in or on the eyepiece, the reflector configured to reflect light received from an object for imaging by said sensor array;

93. The head mounted display of Example 92, wherein said reflector comprises a hot mirror.

94. The head mounted display of Example 92 or 93, wherein said reflector is configured to reflect light of a first range of infrared (IR) or near-IR wavelengths while transmitting light of a second range of visible wavelengths.

95. The head mounted display of any of Examples 92-94, wherein said reflector is formed on at least one of said plurality of layers.

96. The head mounted display of any of Examples 92-95, wherein said reflector comprises one of said plurality of layers.

97. The head mounted display of any of Examples 92-96, wherein said reflector comprises an optical coating.

98. The head mounted display of any of Examples 92-97, wherein said reflector is disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

99. The head mounted display of any of the Examples 87-98, further comprising an off-axis optical element disposed in or on the eyepiece, the off-axis optical element is configured to receive light reflected from a reflector and direct at least of portion of the light toward the at least one imaging lens.

100. The head mounted display of Example 99, wherein said off-axis optical element is disposed in an optical path between said reflector and said at least one imaging lens.

101. The head mounted display of Example 99 or 100, wherein said reflector is disposed on a first layer of said plurality of layers, said off-axis optical element is disposed a second layer of said plurality of layers, and said sensor array is disposed on a third layer of said plurality of layers, said second layer disposed between said first and third layers.

102. The head mounted display of Example 101, wherein said at least a portion of said first, second, and third layers are transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

103. The head mounted display of any of Examples 99-102, wherein said off-axis optical element comprises a diffractive optical element (DOE) or diffraction grating.

104. The head mounted display of any of the Examples 99-103, wherein said off-axis optical element comprises an off-axis diffractive optical element (DOE), an off-axis diffraction grating, an off-axis holographic mirror (OAHM), an off-axis volumetric diffractive optical element (OAVDOE), or an off-axis cholesteric liquid crystal diffraction grating (OACLCG).

105. The head mounted display of any of the Examples 99-104, wherein said off-axis optical element has optical power.

106. The head mounted display of any of any of the Examples 99-105, wherein at least a portion of said off-axis optical element is imprinted on one of the layers of the plurality of layers of the eyepiece.

107. The head mounted display of any of the Examples 87-106, wherein the sensor array is a forward facing camera configured to image at least part of the object based at least in part on light received from a reflector, which is disposed forward of said sensor array, the at least part of the object being disposed rearward of said sensor array and comprising at least one of: the eye of the user, a part of the eye, or a portion of tissue surrounding the eye.

108. The head mounted display of any of the Examples 77-107, further comprising a light source emitting light of a first range of wavelengths toward at least one of: the eye of the user, a part of the eye, or a portion of tissue surrounding the eye.

109. The head mounted display of Example 108, wherein the first range of wavelengths comprises light in at least one of the infrared (IR) or near-IR spectrum.

110. The head mounted display of any of the Examples 77-109, wherein the head mounted display is configured to track the gaze of the user based on images of the at least one of: the eye of the user, the part of the eye, or the portion of tissue surrounding the eye.

111. The head mounted display of any of the Examples 77-106, wherein the sensor array is configured to image an object in the environment in front of said eyepiece.

112. The head mounted display of any of Examples 87-106 and 111, wherein the sensory array is a backward facing camera configured to image at least part of the object based at least in part on light received from a reflector, which is rearward of said sensor array, the at least part of the object disposed forward of the sensor array in the environment in front of the user.

113. The head mounted display of any of Examples 77-112, wherein head mounted display comprises eyewear.

114. A head mounted display comprising:
a frame configured to be supported on the head of the user;
an eyepiece coupled to said frame and configured to be disposed in front of an eye of the user, said eyepiece comprising a plurality of layers disposed forward said eye;
an image injection device configured to provide image content to the eyepiece for viewing by the user;
an imager comprising a sensor array and wafer scale imaging optics integrated in or on the eyepiece; and
a reflector disposed in or on the eyepiece, the reflector configured to reflect light received from an object for imaging by said imager,
wherein said wafer scale imaging optics are not included in a housing that excludes said eyepiece.

115. The head mounted display of Example 114, wherein said plurality of layers of said eyepiece comprises a plurality of planar layers, and said imager is tilted with respect to a normal to said planar layers.

116. The head mounted display of Examples 114 or 115, wherein said imager is tilted in the direction of the reflector.

117. The head mounted display of any of Examples 114-116, wherein said imager is tilted to face the reflector and receive light therefrom.

118. The head mounted display of any of Examples 114-117, wherein said imager is tilted with respect to the forward direction.

119. The head mounted display of any of Examples 114-118, wherein the said eyepiece is configured to direct light from said image injection device to said user's eye to present image content to said user.

120. The head mounted display of any of Examples 114-119, wherein the plurality of layers comprises one or more waveguides configured to receive light from said image injection device and guide at least a portion of the light therein by total internal reflection to provide image content to the user.

121. The head mounted display of any of Examples 114-120, wherein the plurality of layers comprises a plurality of waveguides, different waveguides arranged to provide different color image content.

122. The head mounted display of any of Examples 114-121, wherein the plurality of layers comprises a plurality of waveguides, different waveguides or groups of waveguides configured to project light into the user's eye to display image content with different amounts of divergences as if projected from different distances from the user's eye.

123. The head mounted display of any of Examples 114-122, wherein the plurality of layers comprises one or more of a cosmetic window, a front lens, a dimmer, a rear lens, an illumination layer, or a prescription lens configured to provide for refractive correction for a user having a refractive deficiency.

124. The head mounted display of any of Examples 114-123, wherein said eyepiece comprises at least a portion that is transparent and disposed at a location in front of the user's eye when the user wears the head mounted display device such that the transparent portion transmits light from a portion of the environment in front of the user and the eyepiece to the user's eye to provide a view of the portion of the environment in front of the user and the eyepiece.

125. The head mounted display of any of Examples 114-124, wherein said plurality of layers comprises at least one glass or plastic layer that is transparent.

126. The head mounted display of any of Examples 114-125, wherein said sensor array comprises a plurality of detector pixels formed on at least one of said layers.

127. The head mounted display of any of Examples 114-126, wherein said sensor array comprises a plurality of detector pixels disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

128. The head mounted display of any of Examples 114-127, wherein said wafer scale imaging optics comprises at least one lens aligned with respect to said sensor array such that light from said reflector passes through said at least one lens to said sensor array to form images thereon.

129. The head mounted display of Example 128, wherein said sensor array is disposed in or on a first layer of said plurality of layers of said eyepiece and said at least one lens is disposed in or on a second different layer of said plurality of layers of said eyepiece.

130. The head mounted display of Example 129, wherein first and second layers each include at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

131. The head mounted display of Example 128, wherein said sensor array is disposed in or on a first side of a layer of said plurality of layers of said eyepiece and said at least on lens is disposed in or on a second opposite side of said layer.

132. The head mounted display of Example 131, wherein said layer on which said sensor array and at least one lens is disposed in or on includes at least a portion thereof which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

133. The head mounted display of any of Examples 128-132, wherein at least one lens comprises a diffractive optical element.

134. The head mounted display of any of Examples 128-132, wherein at least one lens comprises at least one refractive lens.

135. The head mounted display of any of Examples 128-134, wherein at least a portion of said at least one lens is imprinted on one of the layers of the plurality of layers of the eyepiece.

136. The head mounted display of any of Examples 114-135, wherein said reflector comprises a hot mirror.

137. The head mounted display of any of Examples 114-136, wherein said reflector is configured to reflect light of a first range of infrared (IR) or near-IR wavelengths while transmitting light of a second range of visible wavelengths.

138. The head mounted display of any of Examples 114-137, wherein said reflector is formed on at least one of said plurality of layers.

139. The head mounted display of any of Examples 114-138, wherein said reflector comprises one of said plurality of layers.

140. The head mounted display of any of Examples 114-139, wherein said reflector comprises an optical coating.

141. The head mounted display of any of Examples 114-140, wherein said reflector is disposed on at least one layer at least a portion of which is transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

142. The head mounted display of any of Examples 114-141, further comprising an off-axis optical element disposed in or on the eyepiece, the off-axis optical element configured to receive light reflected from the reflector and direct at least of portion of the light toward the imager.

143. The head mounted display of Example 142, wherein said off-axis optical element is disposed in an optical path between said reflector and said imager.

144. The head mounted display of any of the Examples 142-143, wherein said reflector is disposed on a first layer of said plurality of layers, said off-axis optical element is disposed a second layer of said plurality of layers, and said sensor array is disposed on a third layer of said plurality of layers, said second layer disposed between said first and third layers.

145. The head mounted display of Example 144, wherein said at least a portion of said first, second, and third layers are transparent and disposed at a location in front of the user's eye when the user wears the head mounted display such that the transparent portion transmits light from a portion of the environment in front of the user and the eyepiece to the user's eye to provide a view of the portion of the environment in front of the user and the eyepiece.

146. The head mounted display of any of the Examples 142-145, wherein said off-axis optical element comprises a diffractive optical element (DOE) or diffraction grating.

147. The head mounted display of any of the Examples 142-146, wherein said off-axis optical element comprises an off-axis diffractive optical element (DOE), an off-axis diffraction grating, an off-axis holographic mirror (OAHM), an off-axis volumetric diffractive optical element (OAVDOE), or an off-axis cholesteric liquid crystal diffraction grating (OACLCG).

148. The head mounted display of any of the Examples 142-147, wherein said off-axis optical element has optical power.

149. The head mounted display of any of the Examples 142-148, wherein at least a portion of said off-axis optical element is imprinted on one of the layers of the plurality of layers of the eyepiece.

150. The head mounted display of any of the Examples 114-149, wherein the imager is a forward facing camera configured to image at least part of the object based at least in part on light received from said reflector, which is disposed forward of said imager, the at least part of the object being disposed rearward of said imager and comprising at least one of: the eye of the user, a part of the eye, or a portion of tissue surrounding the eye.

151. The head mounted display of any of Examples 114-150, further comprising a light source emitting light of a first range of wavelengths toward at least one of: the eye of the user, a part of the eye, or a portion of tissue surrounding the eye.

152. The head mounted display of Example 151, wherein the first range of wavelengths comprises light in at least one of the infrared (IR) or near-IR spectrum.

153. The head mounted display of any of Examples 114-152, wherein the head mounted display is configured to track the gaze of the user based on images of the at least one of: the eye of the user, the part of the eye, or the portion of tissue surrounding the eye.

154. The head mounted display of any of Examples 114-149, wherein the imager is configured to image an object in the environment in front of said eyepiece.

155. The head mounted display of Example 154, wherein the imager is a backward facing camera configured to image at least part of the object based at least in part on light received from said reflector, which is rearward of said imager, the at least part of the object disposed forward of the imager in the environment in front of the user.

156. The head mounted display of any of Examples 114-155, wherein head mounted display comprises eyewear.

### ADDITIONAL CONSIDERATIONS

In the embodiments described above, the optical arrangements have been described in the context of imaging display systems and, more particularly, augmented reality display systems. It will be understood, however, that the principles and advantages of the optical arrangements can be used for other head-mounted display, optical systems, apparatus, or methods. In the foregoing, it will be appreciated that any feature of any one of the embodiments can be combined and/or substituted with any other feature of any other one of the embodiments.

Unless the context clearly requires otherwise, throughout the description and the claims, the words “comprise,” “comprising,” “include,” “including,” “have” and “having” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Depending on the context, “coupled” or “connected” may refer to an optical coupling or optical connection such that light is coupled or connected from one optical element to another optical element. Additionally, the words “herein,” “above,” “below,” “infra,” “supra,” and words of similar import, when used in this application, shall refer to this application as a whole and not to any particular portions of this application. Where the context permits, words in the above Detailed Description using the singular or plural number may also include the plural or singular number, respectively. The word “or” in reference to a list of two or more items is an inclusive (rather than an exclusive) “or”, and “or” covers all of the following interpretations of the word: any of the items in the list, all of the items in the list, and any combination of one or more of the items in the list, and does not exclude other items being added to the list. In addition, the articles “a,” “an,” and “the” as used in this application and the appended claims are to be construed to mean “one or more” or “at least one” unless specified otherwise.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or whether these features, elements and/or states are included or are to be performed in any particular embodiment.

While certain embodiments have been described, these embodiments have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while blocks are presented in a given arrangement, alternative embodiments may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various embodiments described above can be combined to provide further embodiments. The various features and processes described above may be implemented independently of one another, or may be combined in various ways. No element or combinations of elements is necessary or indispensable for all embodiments. All suitable combinations and subcombinations of features of this disclosure are intended to fall within the scope of this disclosure.

1. A head mounted display comprising:
a frame configured to be supported on the head of the user;
an eyepiece coupled to said frame and configured to be disposed in front of an eye of the user, said eyepiece comprising a plurality of layers;
an image injection device configured to provide image content to the eyepiece for viewing by the user;
a sensor array integrated in or on the eyepiece;
a reflector disposed in or on the eyepiece, the reflector configured to reflect light received from an object for imaging by said sensor array; and
a transmissive diffractive or transmissive refractive optical element disposed in or on the eyepiece, the transmissive diffractive or transmissive refractive optical element configured to receive light reflected from the reflector and diffract or refract at least a portion of the light toward the sensor array.

2. The head mounted display of claim 1, wherein said sensor array comprises a plurality of detector pixels formed on at least one of said layers.

3. The head mounted display of claim 1, wherein said sensor array comprises wafer scale optics.

4. The head mounted display of claim 1, wherein said diffractive or refractive optical element comprises at least one lens aligned with respect to said sensor array such that light from said reflector passes through said at least one lens to said sensor array to form images thereon.

5. The head mounted display of claim 4, wherein said sensor array is disposed in or on a first layer of said plurality of layers of said eyepiece and said at least one lens is disposed in or on a second different layer of said plurality of layers of said eyepiece.

6. The head mounted display of claim 4, wherein said sensor array is disposed in or on a first side of a layer of said plurality of layers of said eyepiece and said at least one lens is disposed in or on a second opposite side of said layer.

7. The head mounted display of claim 4, wherein at least one lens comprises wafer scale optics.

8. The head mounted display of claim 1, wherein said reflector comprises a hot mirror.

9. The head mounted display of claim 1, wherein said reflector is configured to reflect light of a first range of infrared (IR) or near-IR wavelengths while transmitting light of a second range of visible wavelengths.

10. The head mounted display of claim 1, wherein said reflector is formed on at least one of said plurality of layers.

11. The head mounted display of claim 1, wherein said reflector comprises one of said plurality of layers.

12. The head mounted display of claim 1, wherein said diffractive or refractive optical element is disposed in an optical path between said reflector and said sensor array.

13. The head mounted display of claim 1, wherein said reflector is disposed on a first layer of said plurality of layers, said diffractive or refractive optical element is disposed on a second layer of said plurality of layers, and said sensor array is disposed on a third layer of said plurality of layers, said second layer disposed between said first and third layers.

14. The head mounted display of claim 1, wherein said diffractive or refractive optical element comprises an off-axis optical element.

15. The head mounted display of claim 1, wherein said diffractive or refractive optical element has optical power.

16. The head mounted display of claim 1, wherein said diffractive or refractive optical element comprises an off-axis lens.

17. The head mounted display of claim 1, wherein at least a portion of said diffractive or refractive optical element is imprinted on one of the layers of the plurality of layers of the eyepiece.

18. The head mounted display of claim 1, wherein the sensor array is a forward facing camera configured to image at least part of the object based at least in part on light received from said reflector, which is disposed forward of said sensor array, the at least part of the object being disposed rearward of said sensor array and comprising at least one of: the eye of the user, a part of the eye, or a portion of tissue surrounding the eye.

19. The head mounted display of claim 1, wherein the sensor array is further configured to image an object in the environment in front of said eyepiece.

20. The head mounted display of claim 19, wherein the sensor array is a backward facing camera configured to image at least part of the object based at least in part on light received from said reflector, which is rearward of said sensor array, the at least part of the object disposed forward of the sensor array in the environment in front of the user.
